(19) United States
(12) Patent Application Publication
(10) Pub. No.: US 2003/0007895 A1
(43) Pub. Date: Jan. 9, 2003

(54) Biochemical analysis unit

(75) Inventor: Hirohiko Tsuzuki, Kanagawa (JP)

Correspondence Address: SUGHRUE MION, PLLC, 2100 Pennsylvania Avenue, NW, Washington, DC, 20037-3213, US

(73) Assignee: FUJI PHOTO FILM CO., LTD.

(21) Appl. No.: 10/173,026

(22) Filed: Jun. 18, 2002

(30) Foreign Application Priority Data
Jun. 19, 2001 (JP) 2001-184583

(51) Int. Cl. G01N 33/543

(52) U.S. Cl. 422/82.08; 436/518

(57) ABSTRACT

A biochemical analysis unit includes a substrate made of a material capable of attenuating radiation energy and light energy and formed with a plurality of holes spaced apart from each other, a plurality of absorptive layers being formed on inner surfaces of the holes. According to the thus constituted biochemical analysis unit, it is possible to prevent noise caused by the scattering of electron beams (β rays) released from a radioactive labeling substance from being generated in biochemical analysis data even in the case of forming spot-like regions selectively containing a radioactive labeling substance in the biochemical analysis unit, superposing the biochemical analysis unit and a stimulable phosphor layer, exposing the stimulable phosphor layer to the radioactive labeling substance, irradiating the stimulable phosphor layer with a stimulating ray to excite the stimulable phosphor, and photoelectrically detecting the stimulated emission released from the stimulable phosphor layer.

### BACKGROUND OF THE INVENTION

The present invention relates to a biochemical analysis unit and, particularly, to a biochemical analysis unit which can prevent noise caused by the scattering of electron beams (β rays) released from a radioactive labeling substance from being generated in biochemical analysis data even in the case of forming spot-like regions of specific binding substances on a substrate at high density, which can specifically bind with a substance derived from a living organism and whose sequence, base length, composition and the like are known, specifically binding the specific binding substances contained in the spot-like regions with a substance derived from a living organism and labeled with a radioactive substance to selectively label the spot-like regions with a radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism; can prevent noise caused by the scattering of chemiluminescence emission from being generated in biochemical analysis data even in the case of forming spot-like regions of specific binding substances on a substrate at high density, which can specifically bind with a substance derived from a living organism and whose sequence, base length, composition and the like are known, specifically binding the specific binding substance contained in the spot-like regions with a substance derived from a living organism and labeled with a labeling substance which generates chemiluminescence emission when it contacts a chemiluminescent substrate to selectively label the spot-like regions with a labeling substance which generates chemiluminescence emission when it contacts a chemiluminescent substrate, thereby obtaining a biochemical analysis unit, bringing the thus obtained biochemical analysis unit into contact with a chemiluminescent substrate, thereby causing it to release chemiluminescence emission, superposing the biochemical analysis unit releasing chemiluminescence emission and a stimulable phosphor layer, exposing the stimulable phosphor layer to the chemiluminescence emission, irradiating the thus exposed stimulable phosphor layer with a stimulating ray to excite the stimulable phosphor, photoelectrically detecting the stimulated emission released from the stimulable phosphor layer to produce biochemical analysis data, and analyzing the substance derived from a living organism; and can prevent noise caused by the scattering of chemiluminescence emission released from a labeling substance which generates chemiluminescence emission when it contacts a chemiluminescent substrate or fluorescence emission released from a fluorescent substance from being generated in biochemical analysis data even in the case of forming spot-like regions of specific binding substances on a substrate at high density, which can specifically bind with a substance derived from a living organism and whose sequence, base length, composition and the like are known, specifically binding the specific binding substance contained in the spot-like regions with a substance derived from a living organism and labeled with, in addition to a radioactive labeling substance or instead of a radioactive labeling substance, a labeling substance which generates chemiluminescence emission when it contacts a chemiluminescent substrate and/or a fluorescent substance to selectively label the spot-like specific binding substances therewith, thereby obtaining a biochemical analysis unit, photoelectrically detecting chemiluminescence emission or fluorescence emission released from the biochemical analysis unit to produce biochemical analysis data, and analyzing the substance derived from a living organism.

### DESCRIPTION OF THE PRIOR ART

An autoradiographic analyzing system using as a detecting material for detecting radiation a stimulable phosphor which can absorb, store and record the energy of radiation when it is irradiated with radiation and which, when it is then stimulated by an electromagnetic wave having a specified wavelength, can release stimulated emission whose light amount corresponds to the amount of radiation with which it was irradiated is known, which comprises the steps of introducing a radioactively labeled substance into an organism, using the organism or a part of the tissue of the organism as a specimen, superposing the specimen and a stimulable phosphor sheet formed with a stimulable phosphor layer for a certain period of time, storing and recording radiation energy in a stimulable phosphor contained in the stimulable phosphor layer, scanning the stimulable phosphor layer with an electromagnetic wave to excite the stimulable phosphor, photoelectrically detecting the stimulated emission released from the stimulable phosphor to produce digital image signals, effecting image processing on the obtained digital image signals, and reproducing an image on displaying means such as a CRT or the like or a photographic film (see, for example, Japanese Patent Publication No. 1-60784, Japanese Patent Publication No. 1-60782, Japanese Patent Publication No. 4-3952 and the like).

Unlike the system using a photographic film, according to the autoradiographic analyzing system using the stimulable phosphor as a detecting material, development, which is chemical processing, becomes unnecessary. Further, it is possible reproduce a desired image by effecting image processing on the obtained image data and effect quantitative analysis using a computer. Use of a stimulable phosphor in these processes is therefore advantageous.

On the other hand, a fluorescence analyzing system using a fluorescent substance as a labeling substance instead of a radioactive labeling substance in the autoradiographic analyzing system is known. According to this system, it is possible to study a genetic sequence, study the expression level of a gene, and to effect separation or identification of protein or estimation of the molecular weight or properties of protein or the like. For example, this system can perform a process including the steps of distributing a plurality of DNA fragments on a gel support by means of electrophoresis after a fluorescent dye was added to a solution containing a plurality of DNA fragments to be distributed, or distributing a plurality of DNA fragments on a gel support containing a fluorescent dye, or dipping a gel support on which a plurality of DNA fragments have been distributed by means of electrophoresis in a solution containing a fluorescent dye, thereby labeling the electrophoresed DNA fragments, exciting the fluorescent dye by a stimulating ray to cause it to release fluorescence emission, detecting the released fluorescence emission to produce an image and detecting the distribution of the DNA fragments on the gel support. This system can also perform a process including the steps of distributing a plurality of DNA fragments on a gel support by means of electrophoresis, denaturing the DNA fragments, transferring at least a part of the denatured DNA fragments onto a transfer support such as a nitrocellulose support by the Southern-blotting method, hybridizing a probe prepared by labeling target DNA and DNA or RNA complementary thereto with the denatured DNA fragments, thereby selectively labeling only the DNA fragments complementary to the probe DNA or probe RNA, exciting the fluorescent dye by a stimulating ray to cause it to release fluorescence emission, detecting the released fluorescence emission to produce an image and detecting the distribution of the target DNA on the transfer support. This system can further perform a process including the steps of preparing a DNA probe complementary to DNA containing a target gene labeled by a labeling substance, hybridizing it with DNA on a transfer support, combining an enzyme with the complementary DNA labeled by a labeling substance, causing the enzyme to contact a fluorescent substance, transforming the fluorescent substance to a fluorescent substance having fluorescence emission releasing property, exciting the thus produced fluorescent substance by a stimulating ray to release fluorescence emission, detecting the fluorescence emission to produce an image and detecting the distribution of the target DNA on the transfer support. This fluorescence detecting system is advantageous in that a genetic sequence or the like can be easily detected without using a radioactive substance.

Similarly, there is known a chemiluminescence detecting system comprising the steps of fixing a substance derived from a living organism such as a protein or a nucleic acid sequence on a support, selectively labeling the substance derived from a living organism with a labeling substance which generates chemiluminescence emission when it contacts a chemiluminescent substrate, contacting the substance derived from a living organism and selectively labeled with the labeling substance and the chemiluminescent substrate, photoelectrically detecting the chemiluminescence emission in the wavelength of visible light generated by the contact of the chemiluminescent substrate and the labeling substance to produce digital image signals, effecting image processing thereon, and reproducing a chemiluminescent image on a display means such as a CRT or a recording material such as a photographic film, thereby obtaining information relating to the high molecular substance such as genetic information

Further, a micro-array analyzing system has been recently developed, which comprises the steps of using a spotting device to drop at different positions on the surface of a carrier such as a slide glass plate, a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substances using a hybridization method or the like with a substance derived from a living organism such as a cell, virus, hormone, tumor marker, enzyme, antibody, antigen, abzyme, other protein, a nuclear acid, cDNA, DNA or mRNA by extraction, isolation or the like and optionally further subjected to chemical processing, chemical modification or the like and which is labeled with a labeling substance such as a fluorescent substance, dye or the like, thereby forming a micro-array, irradiating the micro-array with a stimulating ray, photoelectrically detecting light such as fluorescence emission released from a labeling substance such as a fluorescent substance, dye or the like, and analyzing the substance derived from a living organism. This micro-array analyzing system is advantageous in that a substance derived from a living organism can be analyzed in a short time period by forming a number of spots of specific binding substances at different positions of the surface of a carrier such as a slide glass plate at high density and hybridizing them with a substance derived from a living organism and labeled with a labeling substance.

In addition, a macro-array analyzing system using a radioactive labeling substance as a labeling substance has been further developed, which comprises the steps of using a spotting device to drop at different positions on the surface of a carrier such as a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substance using a hybridization method or the like with a substance derived from a living organism such as a cell, virus, hormone, tumor marker, enzyme, antibody, antigen, abzyme, other protein, a nuclear acid, cDNA, DNA or mRNA by extraction, isolation or the like and optionally further subjected to chemical processing, chemical modification or the like and which is labeled with a radioactive labeling substance, thereby forming a macro-array, superposing the macro-array and a stimulable phosphor sheet formed with a stimulable phosphor layer, exposing the stimulable phosphor layer to a radioactive labeling substance, irradiating the stimulable phosphor layer with a stimulating ray to excite the stimulable phosphor, photoelectrically detecting the stimulated emission released from the stimulable phosphor to produce biochemical analysis data, and analyzing the substance derived from a living organism.

However, in the macro-array analyzing system using a radioactive labeling substance as a labeling substance, when the stimulable phosphor layer is exposed to a radioactive labeling substance, since the radiation energy of the radioactive labeling substance contained in spot-like regions formed on the surface of a carrier such as a membrane filter is very large, electron beams (β rays) released from the radioactive labeling substance contained in the individual spot-like regions are scattered in the carrier such as a membrane filter, thereby impinging on regions of the stimulable phosphor layer that should be exposed only to the radioactive labeling substance contained in neighboring spot-like regions, or electron beams (β rays) released from the radioactive labeling substance adhering to the surface of the carrier such as a membrane filter between neighboring spot-like regions impinge on the stimulable phosphor layer, to generate noise in biochemical analysis data produced by photoelectrically detecting stimulated emission, thus making data of neighboring spot-like regions hard to separate and lowering resolution, and to lower the accuracy of biochemical analysis when a substance derived from a living organism is analyzed by quantifying the radiation amount of each spot. The degradation of the resolution and accuracy of biochemical analysis is particularly pronounced when spots are formed close to each other at high density.

In order to solve these problems by preventing noise caused by the scattering of electron beams (β rays) released from radioactive labeling substance contained in neighboring spot-like regions, it is inevitably required to increase the distance between neighboring spot-like regions and this makes the density of the spot-like regions lower and the test efficiency lower.

Further, in the field of biochemical analysis, it is often required to analyze a substance derived from a living organism by forming a plurality of spot-like regions containing specific binding substances spot-like formed at different positions on the surface of a carrier such as a membrane filter or the like, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, specifically binding, using a hybridization method or the like, the specific binding substances contained in the plurality of spot-like regions with a substance derived from a living organism labeled with, in addition to a radioactive labeling substance or instead of a radioactive labeling substance, a labeling substance which generates chemiluminescence emission when it contacts a chemiluminescent substrate and/or a fluorescent substance, thereby selectively labeling the plurality of spot-like regions, causing the plurality of spot-like regions to contact a chemiluminescent substrate, thereby photoelectrically detecting the chemiluminescence emission in the wavelength of visible light, or irradiating the plurality of spot-like regions with a stimulating ray, thereby photoelectrically detecting fluorescence emission released from a fluorescent substance. In these cases, chemiluminescence emission or fluorescence emission released from the plurality of spot-like regions is scattered in the carrier such as a membrane filter or chemiluminescence emission or fluorescence emission released from any particular spot-like region is scattered and mixed with chemiluminescence emission or fluorescence emission released from neighboring spot-like regions, thereby generating noise in biochemical analysis data produced by photoelectrically detecting chemiluminescence emission or fluorescence emission.

Furthermore, in the field of biochemical analysis, it is often required to analyze a substance derived from a living organism by forming a plurality of spot-like regions containing specific binding substances at different positions on the surface of a carrier such as a membrane filter or the like, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, specifically binding, using a hybridization method or the like, the specific binding substances contained in the plurality of spot-like regions with a substance derived from a living organism labeled with a labeling substance which generates chemiluminescence emission when it contacts a chemiluminescent substrate, thereby selectively labeling the plurality of spot-like regions, causing the plurality of spot-like regions to come into contact with a chemiluminescent substrate, exposing a stimulable phosphor layer to chemiluminescence emission in the wavelength of visible light generated by the contact of the chemiluminescent substance and the labeling substance, thereby storing the energy of chemiluminescence emission in the stimulable phosphor layer, irradiating the stimulable phosphor layer with a stimulating ray, and photoelectrically detecting stimulated emission released from the stimulable phosphor layer, thereby effecting biochemical analysis. In this case, chemiluminescence emission released from any particular spot-like region is scattered in the carrier such as a membrane filter, thereby impinging on regions of the stimulable phosphor layer that should be exposed only to the chemiluminescence emission released from neighboring spot-like regions to generate noise in biochemical analysis data produced by photoelectrically detecting stimulated emission, thus making data of neighboring spot-like regions hard to separate and lowering resolution, and to lower the quantitative characteristics of biochemical analysis data.

### SUMMARY OF THE INVENTION

It is therefore an object of the present invention to provide a biochemical analysis unit which can prevent noise caused by the scattering of electron beams (β rays) released from a radioactive labeling substance from being generated in biochemical analysis data even in the case of forming spot-like regions of specific binding substances on a substrate at high density, which can specifically bind with a substance derived from a living organism and whose sequence, base length, composition and the like are known, specifically binding the specific binding substances contained in the spot-like regions with a substance derived from a living organism and labeled with a radioactive substance to selectively label the spot-like regions with a radioactive substance, thereby obtaining a biochemical analysis unit, superposing the thus obtained biochemical analysis unit and a stimulable phosphor layer, exposing the stimulable phosphor layer to the radioactive labeling substance, irradiating the thus exposed stimulable phosphor layer with a stimulating ray to excite the stimulable phosphor, photoelectrically detecting the stimulated emission released from the stimulable phosphor layer to produce biochemical analysis data, and analyzing the substance derived from a living organism.

It is another object of the present invention is to provide a biochemical analysis unit which can prevent noise caused by the scattering of chemiluminescence emission from being generated in biochemical analysis data even in the case of forming spot-like regions of specific binding substances on a substrate at high density, which can specifically bind with a substance derived from a living organism and whose sequence, base length, composition and the like are known, specifically binding the specific binding substance contained in the spot-like regions with a substance derived from a living organism and labeled with a labeling substance which generates chemiluminescence emission when it contacts a chemiluminescent substrate to selectively label the spot-like regions with a labeling substance which generates chemiluminescence emission when it contacts a chemiluminescent substrate, thereby obtaining a biochemical analysis unit, bringing the thus obtained biochemical analysis unit into contact with a chemiluminescent substrate, thereby causing it to release chemiluminescence emission, superposing the biochemical analysis unit releasing chemiluminescence emission and a stimulable phosphor layer, exposing the stimulable phosphor layer to the chemiluminescence emission, irradiating the thus exposed stimulable phosphor layer with a stimulating ray to excite the stimulable phosphor, photoelectrically detecting the stimulated emission released from the stimulable phosphor layer to produce biochemical analysis data, and analyzing the substance derived from a living organism.

It is a further object of the present invention is to provide a biochemical analysis unit which can prevent noise caused by the scattering of chemiluminescence emission released from a labeling substance which generates chemiluminescence emission when it contacts a chemiluminescent substrate or fluorescence emission released from a fluorescent substance from being generated in biochemical analysis data even in the case of forming spot-like regions of specific binding substances on a substrate at high density, which can specifically bind with a substance derived from a living organism and whose sequence, base length, composition and the like are known, specifically binding the specific binding substance contained in the spot-like regions with a substance derived from a living organism and labeled with, in addition to a radioactive labeling substance or instead of a radioactive labeling substance, a labeling substance which generates chemiluminescence emission when it contacts a chemiluminescent substrate and/or a fluorescent substance to selectively label the spot-like specific binding substances therewith, thereby obtaining a biochemical analysis unit, photoelectrically detecting chemiluminescence emission or fluorescence emission released from the biochemical analysis unit to produce biochemical analysis data, and analyzing the substance derived from a living organism.

The above other objects of the present invention can be accomplished by a biochemical analysis unit including a substrate made of a material capable of attenuating radiation energy and/or light energy and formed with a plurality of holes spaced apart from each other, a plurality of absorptive layers being formed on inner surfaces of the holes.

In one mode of use of the biochemical analysis unit according to this aspect of the present invention, the plurality of absorptive layers are formed on the inner surfaces of the plurality of holes formed to be spaced apart from each other in the substrate made of a material capable of attenuating radiation energy, specific binding substances, which can specifically bind with a substance derived from a living organism and whose sequence, base length, composition and the like are known, are spotted in the plurality of holes formed in the biochemical analysis unit, thereby being absorbed in the plurality of absorption layers formed on the inner surfaces of the plurality of holes, and a substance derived from a living organism and labeled with a radioactive substance is specifically bound, using a hybridization method or the like, with the specific binding substances absorbed in the plurality of absorptive layers, thereby selectively labeling the plurality of absorptive layers therewith. The biochemical analysis unit is then disposed so as to face a stimulable phosphor layer, thereby exposing the stimulable phosphor layer to the radioactive labeling substance absorbed in the plurality of absorptive layers. At this time, since electron beams (β rays) released from the radioactive labeling substance absorbed in the individual absorptive layers are attenuated by the substrate capable of attenuating radiation energy, electron beams (β rays) can be effectively prevented from scattering in the substrate of the biochemical analysis unit and thereby exposing the region of the stimulable phosphor layer which each absorptive layer faces to electron beams (β rays) that are released from neighboring absorptive layers and scattered in the substrate. As a result, it can be ensured that only regions of the stimulable phosphor layer facing the individual absorptive layers are selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy.

In another mode of use of the biochemical analysis unit according to this aspect of the present invention, the plurality of absorptive layers are formed on the inner surfaces of the plurality of holes formed to be spaced apart from each other in the substrate made of a material capable of attenuating light energy, specific binding substances, which can specifically bind with a substance derived from a living organism and whose sequence, base length, composition and the like are known, are spotted in the plurality of holes formed in the biochemical analysis unit, thereby being absorbed in the plurality of absorption layers formed on the inner surfaces of the plurality of holes, and a substance derived from a living organism and labeled with a labeling substance which generates chemiluminescence emission when it contacts a chemiluminescent substrate is specifically bound, using a hybridization method or the like, with the specific binding substances absorbed in the plurality of absorptive layers, thereby selectively labeling the plurality of absorptive layers therewith. The biochemical analysis unit is then brought into contact with a chemiluminescent substrate, thereby causing the plurality of spot-like regions to selectively release chemiluminescence emission and disposed so as to face a stimulable phosphor layer, thereby exposing the stimulable phosphor layer to the chemiluminescence emission. At this time, since chemiluminescence emission released from the individual absorptive layers are attenuated by the substrate capable of attenuating light energy, chemiluminescence emission can be effectively prevented from scattering in the substrate of the biochemical analysis unit and thereby exposing the region of the stimulable phosphor layer which each absorptive layer faces to chemiluminescence emission that are released from neighboring absorptive layers and scattered in the substrate. As a result, it can be ensured that only regions of the stimulable phosphor layer facing the individual absorptive layers are selectively exposed to chemiluminescence emission. Therefore, it is possible to efficiently prevent noise caused by the scattering of chemiluminescence emission from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy.

In a further mode of use of the biochemical analysis unit according to this aspect of the present invention, the plurality of absorptive layers are formed on the inner surfaces of the plurality of holes formed to be spaced apart from each other in the substrate made of a material capable of attenuating light energy, specific binding substances, which can specifically bind with a substance derived from a living organism and whose sequence, base length, composition and the like are known, are spotted in the plurality of holes formed in the biochemical analysis unit, thereby being absorbed in the plurality of absorption layers formed on the inner surfaces of the plurality of holes, and a substance derived from a living organism and labeled with a labeling substance which generates chemiluminescence emission when it contacts a chemiluminescent substrate and/or a fluorescent substance, instead of with a radioactive labeling substance, is specifically bound, using a hybridization method or the like, with the specific binding substances absorbed in the plurality of absorptive layers, thereby selectively labeling the plurality of absorptive layers therewith. Biochemical analysis data are then produced by photoelectrically detecting chemiluminescence emission generated by the contact of a chemiluminescent substrate and the labeling substance in response to contact of the biochemical analysis unit and the chemiluminescent substrate or fluorescence emission released from the fluorescent substance in response to irradiating the biochemical analysis unit with a stimulating ray. At this time, since chemiluminescence emission or fluorescence emission is attenuated by the substrate capable of attenuating light energy, chemiluminescence emission or fluorescence emission released from the absorptive layers can be effectively prevented from scattering in the substrate of the biochemical analysis unit. Therefore, it is possible to efficiently prevent noise caused by the scattering chemiluminescence emission or fluorescence emission from being generated in biochemical analysis data produced by photoelectrically detecting chemiluminescence emission or fluorescence emission.

In a further mode of use of the biochemical analysis unit according to this aspect of the present invention, the plurality of absorptive layers are formed on the inner surfaces of the plurality of holes formed to be spaced apart from each other in the substrate made of a material capable of attenuating radiation energy and light energy, specific binding substances, which can specifically bind with a substance derived from a living organism and whose sequence, base length, composition and the like are known, are spotted in the plurality of holes formed in the biochemical analysis unit, thereby being absorbed in the plurality of absorption layers formed on the inner surfaces of the plurality of holes, and a substance derived from a living organism and labeled with a labeling substance which generates chemiluminescence emission when it contacts a chemiluminescent substrate and/or a fluorescent substance, in addition to a radioactive labeling substance, is specifically bound, using a hybridization method or the like, with the specific binding substances absorbed in the plurality of absorptive layers, thereby selectively labeling the plurality of absorptive layers therewith. In the case where the biochemical analysis unit is then disposed so as to face a stimulable phosphor layer, thereby exposing the stimulable phosphor layer to the radioactive labeling substance absorbed in the absorptive layers, since electron beams (β rays) released from the radioactive labeling substance absorbed in the individual absorptive layers are attenuated by the substrate of the biochemical analysis unit capable of attenuating radiation energy and light energy, electron beams (β rays) can be effectively prevented from scattering in the substrate, thereby enabling only regions of the stimulable phosphor layer facing the individual absorptive layers to be selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy. On the other hand, when biochemical analysis data are produced by photoelectrically detecting chemiluminescence emission generated by the contact of a chemiluminescent substrate and the labeling substance in response to contact of the biochemical analysis unit and the chemiluminescent substrate or fluorescence emission released from the fluorescent substance in response to irradiating the biochemical analysis unit with a stimulating ray, since the substrate of the biochemical analysis unit is made of a material capable of attenuating radiation energy and light energy, chemiluminescence emission or fluorescence emission can be effectively prevented from scattering in the substrate. Therefore, it is possible to efficiently prevent noise caused by the scattering of chemiluminescence emission or fluorescence emission from being generated in biochemical analysis data produced by photoelectrically detecting chemiluminescence emission or fluorescence emission.

The above and other objects of the present invention can be also accomplished by a biochemical analysis unit including a substrate made of a material capable of attenuating radiation energy and/or light energy and formed with a plurality of holes spaced apart from each other, a plurality of absorptive layers being formed on inner surfaces of the plurality of holes formed in the substrate and being selectively labeled with at least one kind of a labeling substance selected from a group consisting a radioactive labeling substance, a fluorescent substance and a labeling substance which generates chemiluminescence emission when it contacts a chemiluminescent substrate.

In one mode of use of the biochemical analysis unit according to this aspect of the present invention, in the case where the substrate of the biochemical analysis unit is made of a material capable of attenuating radiation energy and the plurality of absorptive layers are selectively labeled with a radioactive labeling substance, when the biochemical analysis unit is disposed so as to face a stimulable phosphor layer, thereby exposing the stimulable phosphor layer to the radioactive labeling substance absorbed in the plurality of absorptive layers, since electron beams (β rays) released from the radioactive labeling substance absorbed in the individual absorptive layers are attenuated by the substrate capable of attenuating radiation energy, electron beams (βrays) can be effectively prevented from scattering in the substrate of the biochemical analysis unit and thereby exposing the region of the stimulable phosphor layer which each absorptive layer faces to electron beams (β rays) that are released from neighboring absorptive layers and scattered in the substrate. As a result, it can be ensured that only regions of the stimulable phosphor layer facing the individual absorptive layers are selectively exposed to electron beams (β rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy.

In another mode of use of the biochemical analysis unit according to this aspect of the present invention, in the case where the substrate of the biochemical analysis unit is made of a material capable of attenuating light energy and the plurality of absorptive layers are selectively labeled with a labeling substance which generates chemiluminescence emission when it contacts a chemiluminescent substrate, when the biochemical analysis unit is brought into contact with a chemiluminescent substrate, thereby causing the plurality of absorptive layers to release chemiluminescence emission and disposed so as to face a stimulable phosphor layer, thereby exposing the stimulable phosphor layer to the chemiluminescence emission, since chemiluminescence emission released from the individual absorptive layers are attenuated by the substrate capable of attenuating light energy, chemiluminescence emission can be effectively prevented from scattering in the substrate of the biochemical analysis unit and thereby exposing the region of the stimulable phosphor layer which each absorptive layer faces to chemiluminescence emission that are released from neighboring absorptive layers and scattered in the substrate. As a result, it can be ensured that only regions of the stimulable phosphor layer facing the individual absorptive layers are selectively exposed to chemiluminescence emission. Therefore, it is possible to efficiently prevent noise caused by the scattering of chemiluminescence emission from being generated in biochemical analysis data produced by irradiating the stimulable phosphor layer exposed to the chemiluminescence emission with a stimulating ray and photoelectrically detecting stimulated emission released from the stimulable phosphor layer and to produce biochemical analysis data having high quantitative accuracy.

In a further mode of use of the biochemical analysis unit according to this aspect of the present invention, in the case where the substrate of the biochemical analysis unit is made of a material capable of attenuating light energy and the plurality of absorptive layers are selectively labeled with a fluorescent substance and/or a labeling substance which generates chemiluminescence emission when it contacts a chemiluminescent substrate, when biochemical analysis data are produced by photoelectrically detecting chemiluminescence emission generated by the contact of a chemiluminescent substrate and the labeling substance in response to contact of the biochemical analysis unit and the chemiluminescent substrate or fluorescence emission released from the fluorescent substance in response to irradiating the biochemical analysis unit with a stimulating ray, since chemiluminescence emission or fluorescence emission is attenuated by the substrate capable of attenuating light energy, chemiluminescence emission or fluorescence emission released from the absorptive layers can be effectively prevented from scattering in the substrate of the biochemical analysis unit. Therefore, it is possible to efficiently prevent noise caused by the scattering of chemiluminescence emission or fluorescence emission from being generated in biochemical analysis data produced by photoelectrically detecting chemiluminescence emission or fluorescence emission.

In a further mode of use of the biochemical analysis unit according to this aspect of the present invention, in the case where the substrate of the biochemical analysis unit is made of a material capable of attenuating radiation energy and light energy and the plurality of absorptive layers are selectively labeled with, in addition to a radioactive labeling substance, a fluorescent substance and/or a labeling substance which generates chemiluminescence emission when it contacts a chemiluminescent substrate, at the time the biochemical analysis unit is disposed so as to face a stimulable phosphor layer, thereby exposing the stimulable phosphor layer to the radioactive labeling substance absorbed in the absorptive layers, since electron beams (β rays) released from the radioactive labeling substance absorbed in the individual absorptive layers are attenuated by the substrate of the biochemical analysis unit capable of attenuating radiation energy and light energy, electron beams (β rays) can be effectively prevented from scattering in the substrate, thereby enabling only regions of the stimulable phosphor layer facing the individual absorptive layers to be selectively exposed to electron beams (B rays). Therefore, it is possible to efficiently prevent noise caused by the scattering of electron beams released from the radioactive labeling substance from being generated in biochemical analysis data produced by irradiating the stimulable phosphor layer exposed to the radioactive labeling substance with a stimulating ray and photoelectrically detecting stimulated emission released from the stimulable phosphor layer and to produce biochemical analysis data having high quantitative accuracy. On the other hand, when biochemical analysis data are produced by photoelectrically detecting chemiluminescence emission generated by the contact of a chemiluminescent substrate and the labeling substance in response to contact of the biochemical analysis unit and the chemiluminescent substrate or fluorescence emission released from the fluorescent substance in response to irradiating the biochemical analysis unit with a stimulating ray, since the substrate of the biochemical analysis unit is made of a material capable of attenuating radiation energy and light energy, chemiluminescence emission or fluorescence emission can be effectively prevented from scattering in the substrate. Therefore, it is possible to efficiently prevent noise caused by the scattering of chemiluminescence emission or fluorescence emission from being generated in biochemical analysis data produced by photoelectrically detecting chemiluminescence emission or fluorescence emission.

In the present invention, the plurality of absorptive layers of the biochemical analysis unit are preferable selectively labeled with a radioactive labeling substance by causing the plurality of absorptive layers to absorb specific binding substances whose sequence, base length, composition and the like are known and specifically binding a substance derived from a living organism and labeled with a radioactive labeling substance with the specific binding substances.

In the present invention, the case where a plurality of absorptive layers of the biochemical analysis unit are selectively labeled with a fluorescent substance as termed herein includes the case where a plurality of absorptive layers are selectively labeled with a fluorescent substance by selectively binding a substance derived from a living organism and labeled with a fluorescent substance with specific binding substances contained in the plurality of absorptive layers and the case where a plurality of absorptive layers are selectively labeled with a fluorescent substance by selectively binding a substance derived from a living organism and labeled with a hapten, binding an antibody for the hapten labeled with an enzyme which generates a fluorescent substance when it contacts a fluorescent substrate with the hapten by an antigen-antibody reaction, and causing the enzyme bound with the hapten to come into contact with the fluorescent substrate to generate a fluorescent substance.

Further, in the present invention, the case where a plurality of absorptive layers of the biochemical analysis unit are selectively labeled with a labeling substance which generates chemiluminescence emission when it contacts a chemiluminescent substrate as termed herein includes the case where a plurality of absorptive layers are selectively labeled with a labeling substance which generates chemiluminescence emission when it contacts a chemiluminescent substrate by selectively binding a substance derived from a living organism and labeled with a labeling substance which generates chemiluminescence emission when it contacts a chemiluminescent substrate and the case where a plurality of absorptive layers are selectively labeled with a labeling substance which generates chemiluminescence emission when it contacts a chemiluminescent substrate by selectively binding a substance derived from a living organism and labeled with a hapten, and binding an antibody for the hapten labeled with an enzyme which generates chemiluminescence emission when it contacts a chemiluminescent substrate with the hapten by an antigen-antibody reaction.

In the present invention, illustrative examples of the combination of hapten and antibody include digoxigenin and anti-digoxigenin antibody, theophylline and anti-theophylline antibody, fluorosein and anti-fluorosein antibody, and the like. Further, the combination of biotin and avidin, antigen and antibody may be utilized instead of the combination of hapten and antibody.

In a preferred aspect of the present invention, the plurality of holes are formed by recesses.

According to this preferred aspect of the present invention, since the plurality of absorptive layers are formed on the inner surfaces of the plurality of recesses formed in the substrate, specific binding substances can be absorbed as a probe in regions having a small volume and, therefore, the rate of a specific binding reaction such as hybridization can be improved. Further, since a solution containing a substance derived from a living organism can be brought into contact with the absorptive layers having a large surface area, it is possible to increase the probability of a substance derived from a living organism as a target associating with the specific binding substances absorbed in the absorptive layers as a probe. Therefore, the efficiency of a specific binding reaction such as hybridization can be markedly improved.

Further, according to this preferred aspect of the present invention, since the specific binding substances are absorbed in the absorptive layers formed on the inner surface of the plurality of recesses in the substrate and the substance derived from a living organism and labeled with the labeling substance is hybridized with the specific binding substances contained in the absorptive layers, when the biochemical analysis unit is to be washed, it is sufficient to wash the absorptive layers formed on the inner surface of the plurality of recesses and since each of the absorptive layers has a large surface area, it is possible to efficiently wash the biochemical analysis unit for reuse.

In another preferred aspect of the present invention, the plurality of holes are formed by through-holes.

According to this preferred aspect of the present invention, since the plurality of absorptive layers are formed on the inner surfaces of the plurality of through-holes formed in the substrate, specific binding substances functioning as a probe can be absorbed in regions having a small volume and, therefore, the rate of a specific binding reaction such as hybridization can be improved. Further, since a solution containing a substance derived from a living organism can be brought into contact with the absorptive layers having a large surface area, it is possible to increase the probability of a substance derived from a living organism as a target associating with the specific binding substances absorbed in the absorptive layers as a probe. Therefore, the efficiency of a specific binding reaction such as hybridization can be markedly improved.

Further, according to this preferred aspect of the present invention, since the specific binding substances are absorbed in the absorptive layers formed on the inner surface of the plurality of through-holes in the substrate and the substance derived from a living organism and labeled with the labeling substance is hybridized with the specific binding substances contained in the absorptive layers, when the biochemical analysis unit is to be washed, it is sufficient to wash the absorptive layers formed on the inner surface of the plurality of through-holes and since each of the absorptive layers has a large surface area, it is possible to efficiently wash the biochemical analysis unit for reuse.

In the present invention, a porous material or a fiber material may be preferably used as the absorptive material for forming the absorptive layers of the biochemical analysis unit. The absorptive layers may be formed by combining a porous material and a fiber material.

In the present invention, a porous material for forming the absorptive layers of the biochemical analysis unit may be any type of an organic material or an inorganic material and may be an organic/inorganic composite material.

In the present invention, an organic porous material used for forming the absorptive layers of the biochemical analysis unit is not particularly limited but a carbon porous material such as an activated carbon or a porous material capable of forming a membrane filter is preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluoride such as polyvinylidene fluoride, polytetrafluoride; and copolymers or composite materials thereof.

In the present invention, an inorganic porous material used for forming the absorptive layers of the biochemical analysis unit is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof.

In the present invention, a fiber material used for forming the absorptive layers of the biochemical analysis unit is not particularly limited. Illustrative examples of fiber materials preferably usable in the present invention include nylons such as nylon-6, nylon-6,6, nylon-4,10; and cellulose derivatives such as nitrocellulose, acetyl cellulose, butyric-acetyl cellulose.

In the present invention, the absorptive layer may be formed using an oxidization process such as an electrolytic process, a plasma process, an arc discharge process or the like; a primer process using a silane coupling agent, titanium coupling agent or the like; and a surface-active agent process or the like.

In another preferred aspect of the present invention, the plurality of absorptive layers are formed by surface-processing the substrate with a surface modifying reforming agent.

In a preferred aspect of the present invention, the surfaces of the plurality of absorptive layers are roughened.

According to this preferred aspect of the present invention, since the surfaces of the plurality of absorptive layers are roughened, each of the absorptive layers has a large surface area and it is therefore possible for each of the absorptive layers to absorb a sufficient amount of a specific binding substance.

In a further preferred aspect of the present invention, the surface of each of the absorptive layers is roughened so as to have a fractal structure.

According to this preferred aspect of the present invention, since the surface of each of the absorptive layers is roughened so as to have a fractal structure, each of the absorptive layers has a hundred times or more the absorptive surface area of one having a smooth surface and it is therefore possible for each of the absorptive layers to absorb a sufficient amount of a specific binding substance.

In a preferred aspect of the present invention, the substance derived from a living organism is specifically bound with specific binding substances by a reaction selected from a group consisting of hybridization, antigen-antibody reaction and receptor-ligand reaction.

In a preferred aspect of the present invention, the material capable of attenuating radiation energy has a property of reducing the energy of radiation to ⅕ or less when the radiation travels in the material by a distance equal to that between neighboring absorptive layers.

In a further preferred aspect of the present invention, the material capable of attenuating radiation energy has a property of reducing the energy of radiation to 1/10 or less when the radiation travels in the material by a distance equal to that between neighboring absorptive layers.

In a further preferred aspect of the present invention, the material capable of attenuating radiation energy has a property of reducing the energy of radiation to 1/50 or less when the radiation travels in the material by a distance equal to that between neighboring absorptive layers.

In a further preferred aspect of the present invention, the material capable of attenuating radiation energy has a property of reducing the energy of radiation to 1/100 or less when the radiation travels in the material by a distance equal to that between neighboring absorptive layers.

In a further preferred aspect of the present invention, the material capable of attenuating radiation energy has a property of reducing the energy of radiation to 1/500 or less when the radiation travels in the material by a distance equal to that between neighboring absorptive layers.

In a further preferred aspect of the present invention, the material capable of attenuating radiation energy has a property of reducing the energy of radiation to 1/1000 or less when the radiation travels in the material by a distance equal to that between neighboring absorptive layers.

In a preferred aspect of the present invention, the material capable of attenuating light energy has a property of reducing the energy of light to ⅕ or less when the light travels in the material by a distance equal to that between neighboring absorptive layers.

In a further preferred aspect of the present invention, the material capable of attenuating light energy has a property of reducing the energy of light to 1/10 or less when the light travels in the material by a distance equal to that between neighboring absorptive layers.

In a further preferred aspect of the present invention, the material capable of attenuating light energy has a property of reducing the energy of light to 1/50 or less when the light travels in the material by a distance equal to that between neighboring absorptive layers.

In a further preferred aspect of the present invention, the material capable of attenuating light energy has a property of reducing the energy of light to 1/100 or less when the light travels in the material by a distance equal to that between neighboring absorptive layers.

In a further preferred aspect of the present invention, the material capable of attenuating light energy has a property of reducing the energy of light to 1/500 or less when the light travels in the material by a distance equal to that between neighboring absorptive layers.

In a further preferred aspect of the present invention, the material capable of attenuating light energy has a property of reducing the energy of light to 1/1000 or less when the light travels in the material by a distance equal to that between neighboring absorptive layers.

In the present invention, the material for forming the substrate of the biochemical analysis unit is not particularly limited but may be any type of inorganic compound material or organic compound material insofar as it can attenuate radiation energy and/or light energy. The substrate of the biochemical analysis unit can preferably be formed of metal material, ceramic material or plastic material.

Illustrative examples of inorganic compound materials preferably usable for forming the substrate of the biochemical analysis unit and capable of attenuating radiation energy and/or light energy in the present invention include metals such as gold, silver, copper, zinc, aluminum, titanium, tantalum, chromium, iron, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. These may have either a monocrystal structure or a polycrystal sintered structure such as amorphous, ceramic or the like.

In the present invention, a high molecular compound is preferably used as an organic compound material preferably usable for forming the substrate of the biochemical analysis unit and capable of attenuating radiation energy and/or light energy. Illustrative examples of high molecular compounds preferably usable for forming the substrate of the biochemical analysis unit in the present invention include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifuluoroethylene; polycarbonate; polyesters such as polyethylene naphthalate, polyethylene terephthalate and the like; nylons such as nylon-6, nylon-6,6, nylon-4,10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Since the capability of attenuating radiation energy generally increases as specific gravity increases, the substrate of the biochemical analysis unit is preferably formed of a compound material or a composite material having specific gravity of 1.0 g/cm³ or more and more preferably formed of a compound material or a composite material having specific gravity of 1.5 g/cm³ to 23 g/cm³.

Since the capability of attenuating light energy generally increases as scattering and/or absorption of light increases, in the case where the substrate of the biochemical analysis unit is made of a material capable of attenuating light energy, the substrate of the biochemical analysis unit preferably has absorbance of 0.3 per cm (thickness) or more and more preferably has absorbance of 1 per cm (thickness) or more. The absorbance can be determined by placing an integrating sphere immediately behind a plate-like member having a thickness of T cm, measuring an amount A of transmitted light at a wavelength of probe light or emission light used for measurement by a spectrophotometer, and calculating A/T. In the present invention, a light scattering substance or a light absorbing substance may be added to the substrate of the biochemical analysis unit in order to improve the capability of attenuating light energy. Particles of a material different from a material forming the substrate of the biochemical analysis unit may be preferably used as a light scattering substance and a pigment or dye may be preferably used as a light absorbing substance.

In a preferred aspect of the present invention, the substrate of the biochemical analysis unit is formed of a flexible material.

According to this preferred aspect of the present invention, since the substrate of the biochemical analysis unit is formed of a flexible material, the biochemical analysis unit can be bent and be brought into contact with a reaction solution such as a hybridization reaction solution, thereby specifically binding specific binding substances with a substance derived from a living organism. Therefore, specific binding substances and a substance derived from a living organism can be specifically bound with each other in a desired manner using a small amount of a reaction solution such as a hybridization reaction solution.

In a preferred aspect of the present invention, the plurality of holes are regularly formed in the substrate of the biochemical analysis unit.

In a preferred aspect of the present invention, the plurality of holes having a substantially circular shape are formed in the substrate of the biochemical analysis unit.

In a preferred aspect of the present invention, the plurality of holes having a substantially rectangular shape are formed in the substrate of the biochemical analysis unit.

In a preferred aspect of the present invention, the substrate of the biochemical analysis unit is formed with 10 or more holes.

In a further preferred aspect of the present invention, the substrate of the biochemical analysis unit is formed with 50 or more holes.

In a further preferred aspect of the present invention, the substrate of the biochemical analysis unit is formed with 100 or more holes.

In a further preferred aspect of the present invention, the substrate of the biochemical analysis unit is formed with 500 or more holes.

In a further preferred aspect of the present invention, the substrate of the biochemical analysis unit is formed with 1,000 or more holes.

In a further preferred aspect of the present invention, the substrate of the biochemical analysis unit is formed with 5,000 or more holes.

In a further preferred aspect of the present invention, the substrate of the biochemical analysis unit is formed with 10,000 or more holes.

In a further preferred aspect of the present invention, the substrate of the biochemical analysis unit is formed with 50,000 or more holes.

In a further preferred aspect of the present invention, the substrate of the biochemical analysis unit is formed with 100,000 or more holes.

In a preferred aspect of the present invention, each of the plurality of holes formed in the substrate of the biochemical analysis unit has a size of less than 5 mm².

In a further preferred aspect of the present invention, each of the plurality of holes formed in the substrate of the biochemical analysis unit has a size of less than 1 mm².

In a further preferred aspect of the present invention, each of the plurality of holes formed in the substrate of the biochemical analysis unit has a size of less than 0.5 mm².

In a further preferred aspect of the present invention, each of the plurality of holes formed in the substrate of the biochemical analysis unit has a size of less than 0.1 mm².

In a further preferred aspect of the present invention, each of the plurality of holes formed in the substrate of the biochemical analysis unit has a size of less than 0.05 mm².

In a further preferred aspect of the present invention, each of the plurality of holes formed in the substrate of the biochemical analysis unit has a size of less than 0.01 mm².

In the present invention, the density of the holes formed in the substrate of the biochemical analysis unit is determined depending upon the material for forming the substrate, the kind of electron beam released from a radioactive substance or the like.

In a preferred aspect of the present invention, the plurality of holes are formed in the substrate of the biochemical analysis unit at a density of 10 or more per cm².

In a further preferred aspect of the present invention, the plurality of holes are formed in the substrate of the biochemical analysis unit at a density of 10 or more per cm².

In a further preferred aspect of the present invention, the plurality of holes are formed in the substrate of the biochemical analysis unit at a density of 50 or more per cm².

In a further preferred aspect of the present invention, the plurality of holes are formed in the substrate of the biochemical analysis unit at a density of 100 or more per cm².

In a further preferred aspect of the present invention, the plurality of holes are formed in the substrate of the biochemical analysis unit at a density of 500 or more per cm².

In a further preferred aspect of the present invention, the plurality of holes are formed in the substrate of the biochemical analysis unit at a density of 1,000 or more per cm².

In a further preferred aspect of the present invention, the plurality of holes are formed in the substrate of the biochemical analysis unit at a density of 5,000 or more per cm².

In a further preferred aspect of the present invention, the plurality of holes are formed in the substrate of the biochemical analysis unit at a density of 10,000 or more per cm².

In a further preferred aspect of the present invention, the plurality of holes are formed in the substrate of the biochemical analysis unit at a density of 50,000 or more per cm².

In a further preferred aspect of the present invention, the plurality of holes are formed in the substrate of the biochemical analysis unit at a density of 100,000 or more per cm².

The above and other objects and features of the present invention will become apparent from the following description made with reference to the accompanying drawings.

### BRIEF DESCRIPTION OF THE DRAWINGS

FIG. 1 is a schematic perspective view showing a biochemical analysis unit which is a preferred embodiment of the present invention.

FIG. 2 is a schematic cross sectional view showing a biochemical analysis unit which is a preferred embodiment of the present invention.

FIG. 3 is a schematic front view showing a spotting device.

FIG. 4 is a schematic longitudinal cross sectional view showing a hybridization reaction vessel.

FIG. 5 is a schematic perspective view showing a stimulable phosphor sheet.

FIG. 6 is a schematic cross-sectional view showing a method for exposing a number of stimulable phosphor layer regions formed in a stimulable phosphor sheet to a radioactive labeling substance contained in a number of absorptive layers formed on the inner surfaces of a number of recesses formed in a substrate of a biochemical analysis unit.

FIG. 7 is a schematic perspective view showing one example of a scanner.

FIG. 8 is a schematic perspective view showing details in the vicinity of a photomultiplier of a scanner shown in FIG. 7.

FIG. 9 is a schematic cross-sectional view taken along a line A-A in FIG. 8.

FIG. 10 is a schematic cross-sectional view taken along a line B-B in FIG. 8.

FIG. 11 is a schematic cross-sectional view taken along a line C-C in FIG. 8.

FIG. 12 is a schematic cross-sectional view taken along a line D-D in FIG. 8.

FIG. 13 is a schematic plan view of a scanning mechanism of an optical head.

FIG. 14 is a block diagram of a control system, an input system, a drive system and a detection system of the scanner shown in FIG. 7.

FIG. 15 is a schematic front view showing a data producing system.

FIG. 16 is a schematic longitudinal cross sectional view showing a cooled CCD camera.

FIG. 17 is a schematic vertical cross sectional view showing a dark box.

FIG. 18 is a block diagram of a personal computer and peripheral devices thereof.

FIG. 19 is a schematic perspective view showing a stimulable phosphor sheet onto which chemiluminescence data recorded in a number of the absorptive layers of the biochemical analysis unit are to be transferred.

FIG. 20 is a schematic perspective view showing a scanner for reading chemiluminescence data recorded in a number of stimulable phosphor layer regions formed in a support of a stimulable phosphor sheet and producing biochemical analysis data.

FIG. 21 is a schematic perspective view showing details in the vicinity of a photomultiplier of a scanner shown in FIG. 20.

FIG. 22 is a schematic cross-sectional view taken along a line E-E in FIG. 21.

FIG. 23 is a schematic partial cross sectional view showing a biochemical analysis unit which is another preferred embodiment of the present invention.

FIG. 24 is a schematic perspective view showing a biochemical analysis unit which is a further preferred embodiment of the present invention.

FIG. 25 is a schematic partial cross sectional view showing a biochemical analysis unit which is a further preferred embodiment of the present invention.

### DESCRIPTION OF THE PREFERRED EMBODIMENTS

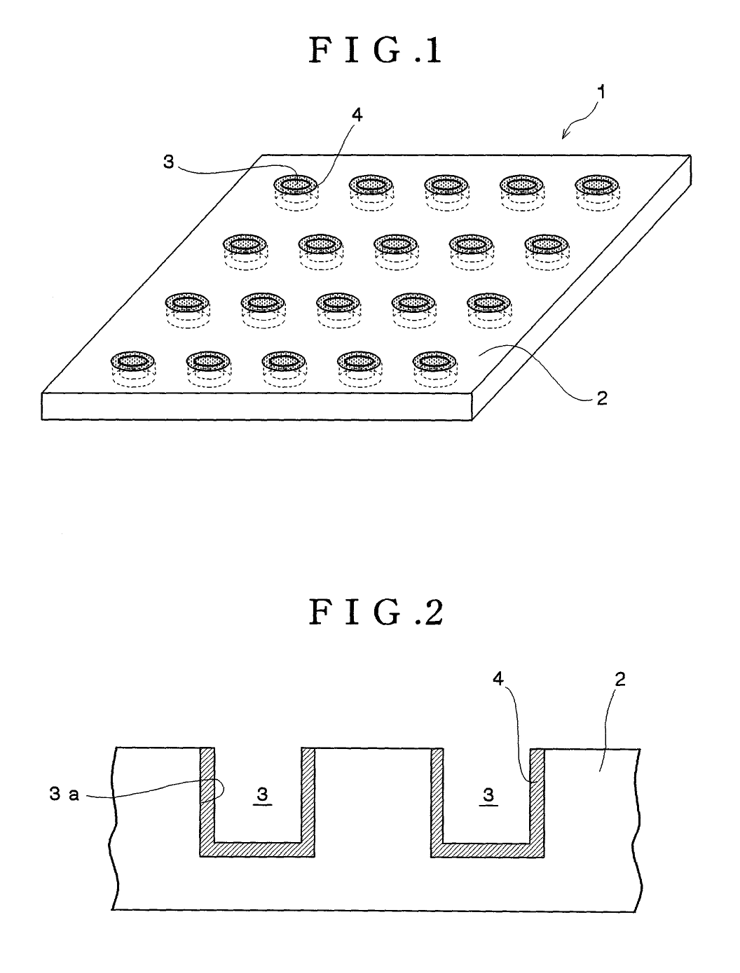

FIG. 1 is a schematic perspective view showing a biochemical analysis unit which is a preferred embodiment of the present invention and FIG. 2 is a schematic cross sectional view thereof.

As shown in FIG. 1, a biochemical analysis unit 1 according to this embodiment includes a substrate 2 made of aluminum capable of attenuating radiation energy and light energy and formed with a number of substantially circular recesses 3 at high density.

As shown in FIG. 2, an absorptive layer 4 is formed of nitrocellulose capable of forming a membrane filter on the inner surface 3a of each of a number of the recesses 3.

Although not accurately shown in FIG. 1, in this embodiment, about 10,000 recesses 3 having a size of about 0.01 mm² are regularly formed at a density of about 5,000 per cm² in the substrate 2.

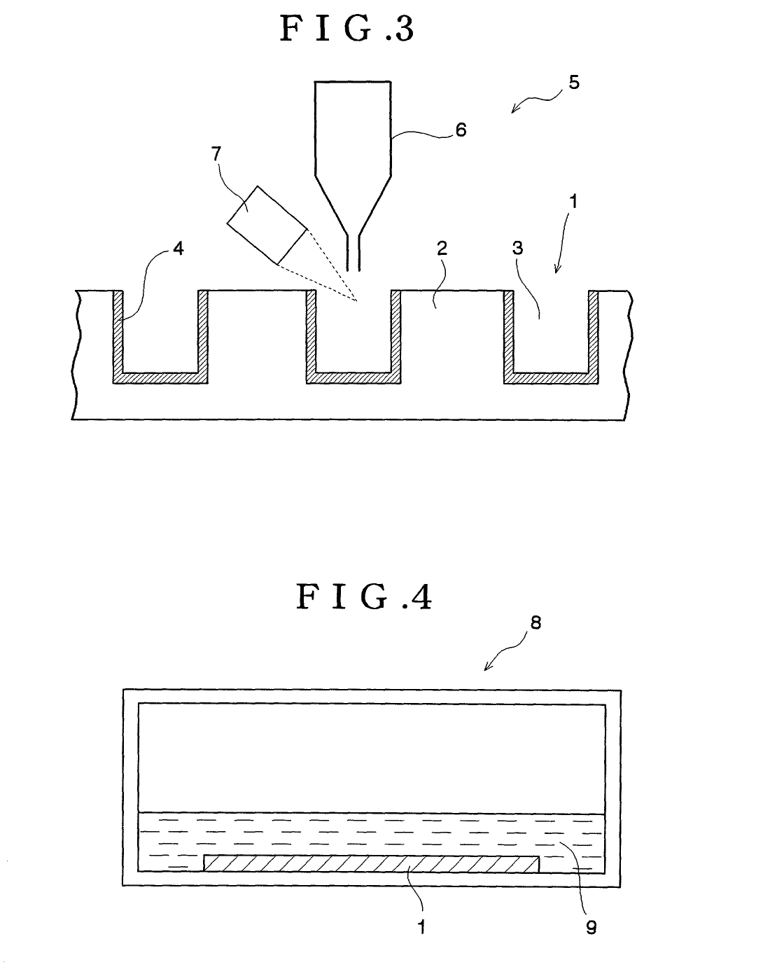

FIG. 3 is a schematic front view showing a spotting device.

As shown in FIG. 3, when biochemical analysis is performed, a solution containing specific binding substances such as a plurality of cDNAs whose sequences are known but differ from each other are spotted using a spotting device 5 into a number of the recesses formed in the substrate 2 of the biochemical analysis unit 1 and the specific binding substances are fixed in a number of the absorptive layers 4 formed in the recesses 3.

As shown in FIG. 2, the spotting device 5 includes an injector 6 for ejecting a solution of specific binding substances toward the biochemical analysis unit 1 and a CCD camera 7 and is constituted so that the solution of specific binding substances such as cDNAs are spotted from the injector 6 when the tip end portion of the injector 6 and the center of the recess 3 into which the solution containing specific binding substances is to be spotted are determined to coincide with each other as a result of viewing them using the CCD camera, thereby ensuring that the solution of specific binding substances can be accurately spotted into a number of the recesses 3 formed with the absorptive layers 3 of the biochemical analysis unit 1.

The specific binding substance spotted in each of the recesses 3 is absorbed in the absorptive layer 4 formed on the recess 3.

FIG. 4 is a schematic longitudinal cross sectional view showing a hybridization reaction vessel.

As shown in FIG. 4, a hybridization reaction vessel 8 is formed to have a substantially rectangular cross section and accommodates a hybridization solution 9 containing as a probe a substance derived from a living organism labeled with a labeling substance.

In the case where a specific binding substance such as cDNA is to be labeled with a radioactive labeling substance, a hybridization solution 9 containing as a probe a substance derived from a living organism and labeled with a radioactive labeling substance is prepared and is accommodated in the hybridization reaction vessel 8.

On the other hand, in the case where a specific binding substance such as cDNA is to be labeled with a labeling substance which generates chemiluminescence emission when it contacts a chemiluminescent substrate, a hybridization solution 9 containing as a probe a substance derived from a living organism and labeled with a labeling substance which generates chemiluminescence emission when it contacts a chemiluminescent substrate is prepared and is accommodated in the hybridization reaction vessel 8.

Further, in the case where a specific binding substance such as cDNA is to be labeled with a fluorescent substance such as a fluorescent dye, a hybridization solution 9 containing as a probe a substance derived from a living organism and labeled with a fluorescent substance such as a fluorescent dye is prepared and is accommodated in the hybridization reaction vessel 8.

It is possible to prepare a hybridization solution 9 containing two or more substances derived from a living organism among a substance derived from a living organism and labeled with a radioactive labeling substance, a substance derived from a living organism and labeled with a labeling substance which generates chemiluminescence emission when it contacts a chemiluminescent substrate and a substance derived from a living organism and labeled with a fluorescent substance such as a fluorescent dye and accommodate it in the hybridization vessel 8. In this embodiment, a hybridization solution 9 containing a substance derived from a living organism and labeled with a radioactive labeling substance. a substance derived from a living organism and labeled with a fluorescent substance such as a fluorescent dye and a substance derived from a living organism and labeled with a labeling substance which generates chemiluminescence emission when it contacts a chemiluminescent substrate is prepared and accommodated in the hybridization reaction vessel 8.

When hybridization is to be performed, the biochemical analysis unit 1 containing specific binding substances such as a plurality of cDNAs absorbed in the absorptive layers 4 formed on the inner surface 3a of a number of the recesses 3 formed in the substrate 1 is accommodated in the hybridization reaction vessel 8.

As a result, specific binding substances absorbed in the absorptive layers 4 formed on the inner surface 3a of a number of the recesses 3 can be selectively hybridized with a substance derived from a living organism, labeled with a radioactive labeling substance and contained in the hybridization reaction solution 9, a substance derived from a living organism, labeled with a fluorescent substance such as a fluorescent dye and contained in the hybridization reaction solution 9, and a substance derived from a living organism, labeled with a labeling substance which generates chemiluminescence emission when it contacts a chemiluminescent substrate and contained in the hybridization reaction solution 9.

In this embodiment, since the specific binding substance functioning as a probe is absorbed in the absorptive layers 4 formed on the inner surface 3a of each of the recesses 3 and absorbed in a region having a smaller volume than that of the case of charging a porous material into the recess 3, the rate of hybridization reaction can be improved. Further, since the hybridization reaction solution 9 can be brought into contact with the absorptive layers 3 having a large surface area, it is possible to increase the probability of a substance derived from a living organism as a target associating with the specific binding substances absorbed in the absorptive layers 4 as a probe. Therefore, the efficiency of hybridization can be markedly improved.

In this manner, radiation data of a radioactive labeling substance, fluorescence data of a fluorescent substance such as a fluorescent dye and chemiluminescence data of the labeling substance which generates chemiluminescence emission when it contacts a chemiluminescent substrate are recorded in the absorptive layers 4 formed on the inner surfaces 3a of a number of the recesses 3 formed in the substrate 2 of the biochemical analysis unit 1.

Fluorescence data recorded in a number of the absorptive layers 4 of the biochemical analysis unit 1 are read by a scanner described later, thereby producing biochemical analysis data.

Further, chemiluminescence data recorded in a number of absorptive regions 4 formed in the biochemical analysis unit 1 are read by a cooled CCD camera of a data producing system described later or transferred onto a stimulable phosphor sheet described later and transferred chemiluminescence data are read by another scanner described later, thereby producing biochemical analysis data.

On the other hand, radiation data recorded in a number of the absorptive layers 4 of the biochemical analysis unit 1 are transferred onto a number of stimulable phosphor layer regions of a stimulable phosphor sheet and read by the scanner described later, thereby producing biochemical analysis data.

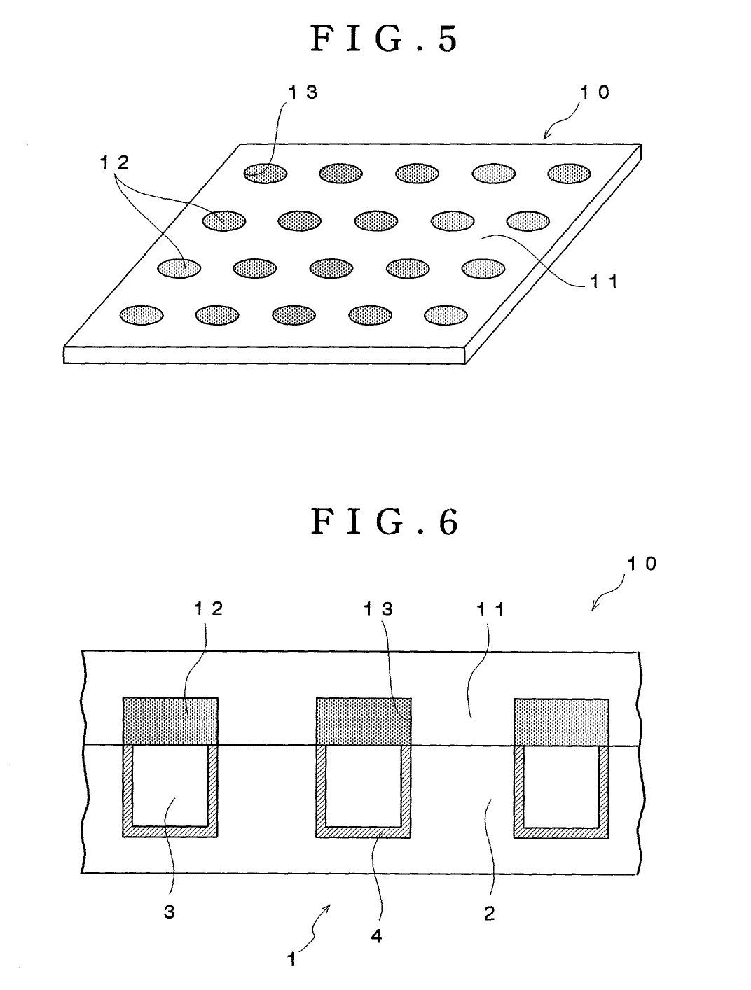

FIG. 5 is a schematic perspective view showing a stimulable phosphor sheet.

As shown in FIG. 5, a stimulable phosphor sheet 10 includes a support 11 formed with a number of recesses 13 on one surface thereof in the same pattern as that of a number of the recesses 3 formed in the biochemical analysis unit 1 and a number of substantially circular stimulable phosphor layer regions 12 are formed by embedding stimulable phosphor in a number of the recesses 13.

In this embodiment, the support 11 is made of stainless steel capable of attenuating radiation energy and a number of recesses 13, namely, a number of the substantially circular stimulable phosphor layer regions 12 are formed in such a manner that each of them has the same diameter as that of the recess 3 formed in the substrate 2 of the biochemical analysis unit 1.

FIG. 6 is a schematic cross-sectional view showing a method for exposing a number of the stimulable phosphor layer regions 12 formed in the stimulable phosphor sheet 10 to a radioactive labeling substance contained in a number of the absorptive layers 4 formed on the inner surfaces 3a of a number of the recesses 3 formed in the substrate 2 of the biochemical analysis unit 1.

As shown in FIG. 6, when the stimulable phosphor layer regions 12 of a stimulable phosphor sheet 10 are to be exposed, the stimulable phosphor sheet 10 is superposed on the biochemical analysis unit 1 in such a manner that a number of the stimulable phosphor layer regions 12 formed in the support 11 of the stimulable phosphor sheet 10 face the corresponding recesses 3 formed in the substrate 2 of the biochemical analysis unit 1.

In this embodiment, since the substrate 2 of the biochemical analysis unit 1 is made of aluminum, the biochemical analysis unit 1 does not stretch or shrink when it is subjected to liquid processing such as hybridization and, therefore, it is possible to easily and accurately superpose the stimulable phosphor sheet 10 on the biochemical analysis unit 1 so that each of the stimulable phosphor layer regions 12 formed in the support 11 of the stimulable phosphor sheet 10 accurately faces the corresponding recess 3 formed with the absorptive layer 4 on the inner surface 3a thereof of the biochemical analysis unit 1, thereby exposing the stimulable phosphor layer regions 12.

In this manner, each of the stimulable phosphor layer regions 12 formed in the support 11 of the stimulable phosphor sheet 10 is kept to be in close contact with the corresponding recess 3 formed with the absorptive layer 4 on the inner surface 3a thereof of the biochemical analysis unit 1 for a predetermined time period, whereby a number of the stimulable phosphor layer regions 12 formed in the support 11 of the stimulable phosphor sheet 10 are exposed to the radioactive labeling substance contained in a number of the absorptive layer 4 formed in the substrate 2 of the biochemical analysis unit 1.

During the exposure operation, electron beams (β rays) are released from the radioactive labeling substance contained in a number of the absorptive layer 4 of the biochemical analysis unit 1. However, since the substrate 2 of the biochemical analysis unit 1 is made of aluminum capable of attenuating radiation energy, electron beams (β rays) released from the radioactive labeling substance contained in the absorptive layers 4 of the biochemical analysis unit 1 can be efficiently prevented from scattering in the substrate 2 of the biochemical analysis unit 1. Further, since the support 11 of the stimulable phosphor sheet 10 is made of stainless steel capable of attenuating radiation energy, electron beams (β rays) released from the radioactive labeling substance contained in the absorptive layers 4 of the biochemical analysis unit 1 can be efficiently prevented from scattering in the support 11 of the stimulable phosphor sheet 10. Therefore, it is possible to prevent the electron beams (β rays) released from the radioactive labeling substance contained in each of the absorptive layers 4 from entering the stimulable phosphor layer regions 12 which the recesses 3 next to the recess 3 in which the absorptive layer 4 is formed face.

Accordingly, it is possible to selectively expose each of a number of the stimulable phosphor layer regions 12 formed in the support 11 of stimulable phosphor sheet 11 to only radioactive labeling substance contained in the absorptive layer 4 formed on the inner surface 3a of the corresponding recess 3 of the biochemical analysis unit 1.

In this manner, radiation data of a radioactive labeling substance are recorded in a number of the stimulable phosphor layer regions 12 formed in the support 11 of the stimulable phosphor sheet 10.

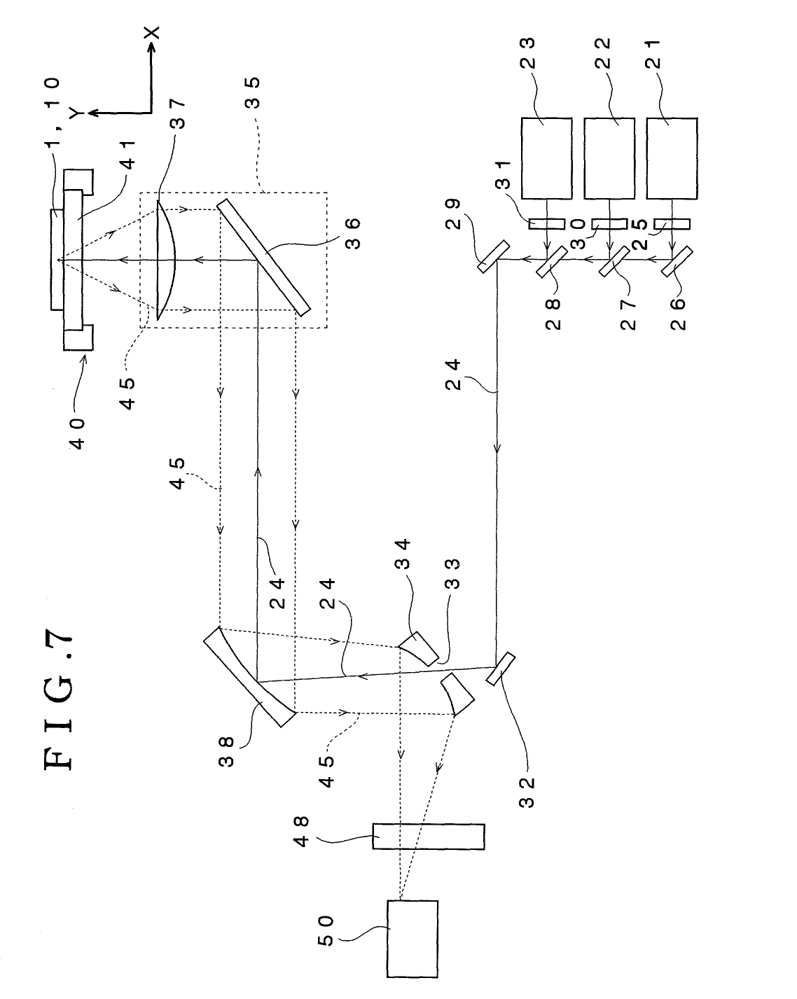

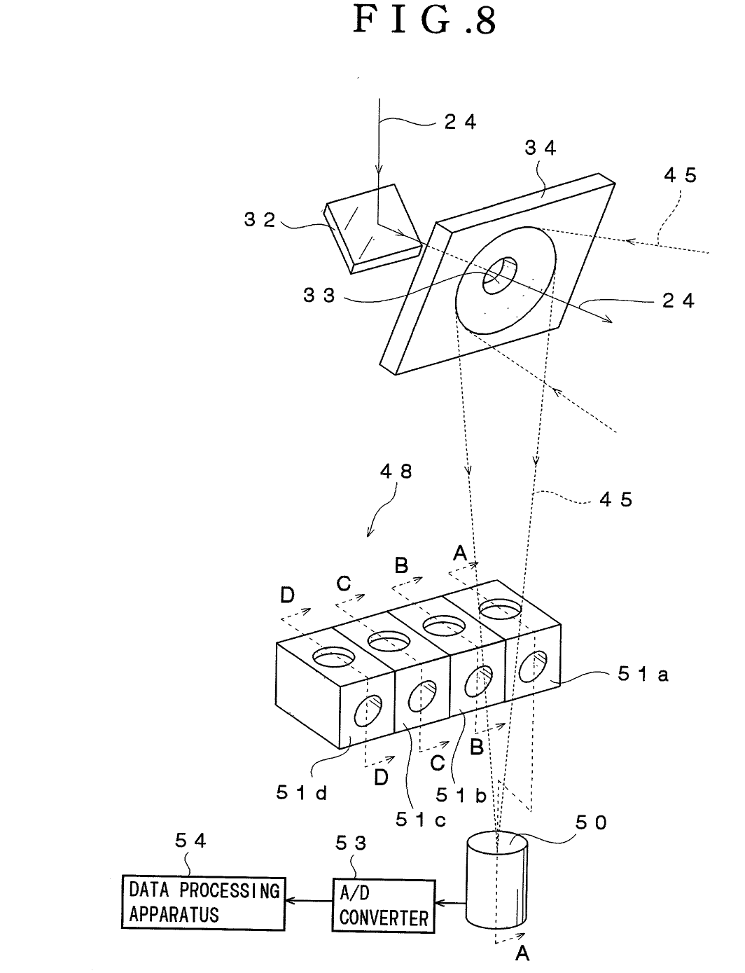

FIG. 7 is a schematic view showing a scanner for reading radiation data of a radioactive labeling substance recorded in a number of the stimulable phosphor layer regions 12 formed in the support 11 of the stimulable phosphor sheet 10 and fluorescence data of a fluorescent substance such as a fluorescent dye recorded in a number of the absorptive layers 4 formed on the inner surfaces 3a of the recesses 4 formed in the substrate 2 of the biochemical analysis unit 1 and producing biochemical analysis data, and FIG. 8 is a schematic perspective view showing details in the vicinity of a photomultiplier.

The scanner shown in FIG. 7 is constituted so as to read radiation data of a radioactive labeling substance recorded in a number of the stimulable phosphor layer regions 12 formed in the support 11 of the stimulable phosphor sheet 10 and fluorescence data of a fluorescent substance such as a fluorescent dye recorded in a number of the absorptive layers 4 formed on the inner surfaces 3a of the recesses 4 formed in the substrate 2 of the biochemical analysis unit 1 and includes a first laser stimulating ray source 21 for emitting a laser beam having a wavelength of 640 nm, a second laser stimulating ray source 22 for emitting a laser beam having a wavelength of 532 nm and a third laser stimulating ray source 23 for emitting a laser beam having a wavelength of 473 nm.

In this embodiment, the first laser stimulating ray source 21 is constituted by a semiconductor laser beam source and the second laser stimulating ray source 22 and the third laser stimulating ray source 23 are constituted by a second harmonic generation element.

A laser beam 24 emitted from the first laser stimulating source 21 passes through a collimator lens 25, thereby being made a parallel beam, and is reflected by a mirror 26. A first dichroic mirror 27 for transmitting light having a wavelength of 640 nm but reflecting light having a wavelength of 532 nm and a second dichroic mirror 28 for transmitting light having a wavelength equal to and longer than 532 nm but reflecting light having a wavelength of 473 nm are provided in the optical path of the laser beam 24 emitted from the first laser stimulating ray source 21. The laser beam 24 emitted from the first laser stimulating ray source 21 and reflected by the mirror 26 passes through the first dichroic mirror 27 and the second dichroic mirror 28 and advances to a mirror 29.

On the other hand, the laser beam 24 emitted from the second laser stimulating ray source 22 passes through a collimator lens 30, thereby being made a parallel beam, and is reflected by the first dichroic mirror 27, thereby changing its direction by 90 degrees. The laser beam 24 then passes through the second dichroic mirror 28 and advances to the mirror 29.

Further, the laser beam 24 emitted from the third laser stimulating ray source 23 passes through a collimator lens 31, thereby being made a parallel beam, and is reflected by the second dichroic mirror 28, thereby changing its direction by 90 degrees. The laser beam 24 then advances to the mirror 29.

The laser beam 24 advancing to the mirror 29 is reflected by the mirror 29 and advances to a mirror 32 to be reflected thereby.

A perforated mirror 34 formed with a hole 33 at the center portion thereof is provided in the optical path of the laser beam 24 reflected by the mirror 32. The laser beam 24 reflected by the mirror 32 passes through the hole 33 of the perforated mirror 34 and advances to a concave mirror 38.

The laser beam 24 advancing to the concave mirror 38 is reflected by the concave mirror 38 and enters an optical head 35.

The optical head 35 includes a mirror 36 and an aspherical lens 37. The laser beam 24 entering the optical head 35 is reflected by the mirror 36 and condensed by the aspherical lens 37 onto the stimulable phosphor sheet 10 or the biochemical analysis unit 1 placed on the glass plate 41 of a stage 40. In FIG. 7, the biochemical analysis unit 1 is placed on the glass plate 41 of the stage so that the surface formed with the recesses 3 faces downward.

When the laser beam 24 impinges on the stimulable phosphor layer region 12 of the stimulable phosphor sheet 10, stimulable phosphor contained in the stimulable phosphor layer region 12 formed in the support 11 of the stimulable phosphor 10 is excited, thereby releasing stimulated emission 45. On the other hand, when the laser beam 24 impinges on the absorptive layer 4 of the biochemical analysis unit 1, a fluorescent dye or the like contained in the absorptive layer 4 formed on the inner surface 3a of the recess 4 is excited, thereby releasing fluorescence emission 45.

The stimulated emission 45 released from the stimulable phosphor layer region 12 of the stimulable phosphor 10 or the fluorescence emission 45 released from the absorptive layer 4 formed on the inner surface 3a of the recess 3 formed in the substrate 2 of the biochemical analysis unit 1 is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 or the fluorescence emission 45 advancing to the concave mirror 38 is reflected by the concave mirror 38 and advances to the perforated mirror 34.

As shown in FIG. 8, the stimulated emission 45 or the fluorescence emission 45 advancing to the perforated mirror 34 is reflected downward by the perforated mirror 34 formed as a concave mirror and advances to a filter unit 48, whereby light having a predetermined wavelength is cut. The stimulated emission 45 or the fluorescence emission 45 then impinges on a photomultiplier 50, thereby being photoelectrically detected.

As shown in FIG. 8, the filter unit 48 is provided with four filter members 51a, 51b, 51c and 51d and is constituted to be laterally movable in FIG. 7 by a motor (not shown).

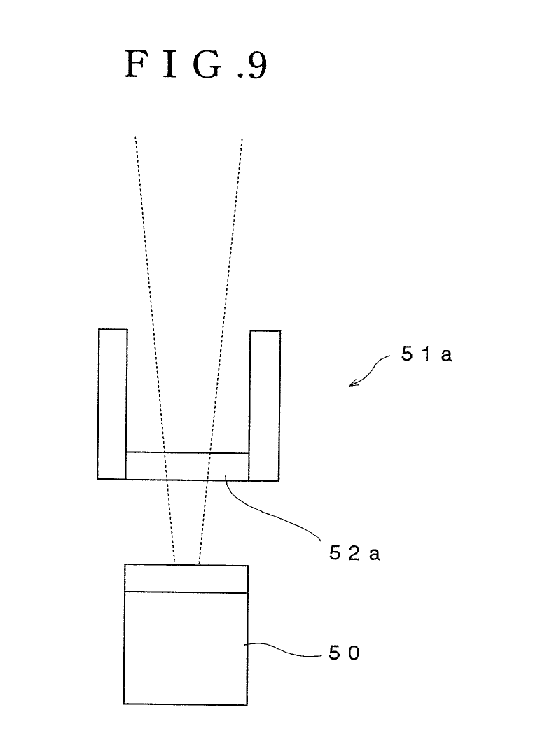

FIG. 9 is a schematic cross-sectional view taken along a line A-A in FIG. 8.

As shown in FIG. 9, the filter member 51a includes a filter 52a and the filter 52a is used for reading fluorescence emission 45 by stimulating a fluorescent substance such as a fluorescent dye contained in a number of the absorptive layers 4 formed on the inner surfaces 3a of the recesses 3 formed in the biochemical analysis unit 1 using the first laser stimulating ray source 21 and has a property of cutting off light having a wavelength of 640 nm but transmitting light having a wavelength longer than 640 nm.

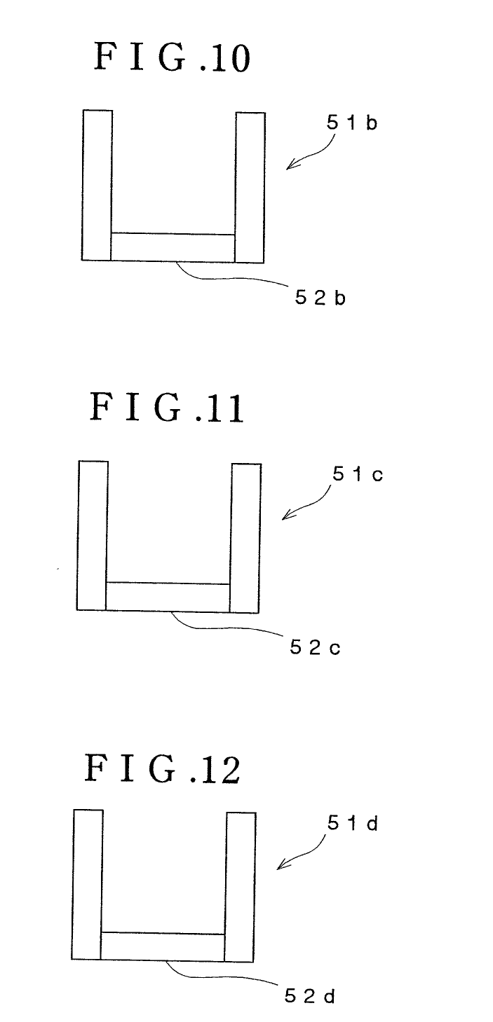

FIG. 10 is a schematic cross-sectional view taken along a line B-B in FIG. 8.

As shown in FIG. 10, the filter member 51b includes a filter 52b and the filter 52b is used for reading fluorescence emission 45 by stimulating a fluorescent substance such as a fluorescent dye contained in a number of the absorptive layers 4 formed on the inner surfaces 3a of the recesses 3 formed in the biochemical analysis unit 1 using the second laser stimulating ray source 22 and has a property of cutting off light having a wavelength of 532 nm but transmitting light having a wavelength longer than 532 nm.

FIG. 11 is a schematic cross-sectional view taken along a line C-C in FIG. 8.

As shown in FIG. 11, the filter member 51c includes a filter 52c and the filter 52c is used for reading fluorescence emission 45 by stimulating a fluorescent substance such as a fluorescent dye contained in a number of the absorptive layers 4 formed on the inner surfaces 3a of the recesses 3 formed in the biochemical analysis unit 1 using the third laser stimulating ray source 23 and has a property of cutting off light having a wavelength of 473 nm but transmitting light having a wavelength longer than 473 nm.

FIG. 12 is a schematic cross-sectional view taken along a line D-D in FIG. 8.

As shown in FIG. 12, the filter member 51d includes a filter 52d and the filter 52d is used for reading stimulated emission released from stimulable phosphor contained in the stimulable phosphor layer 12 formed in the support 11 of the stimulable phosphor sheet 10 upon being stimulated using the first laser stimulating ray source 1 and has a property of transmitting only light having a wavelength corresponding to that of stimulated emission emitted from stimulable phosphor and cutting off light having a wavelength of 640 nm.

Therefore, in accordance with the kind of a stimulating ray source to be used, one of these filter members 51a, 51b, 51c, 51d is selectively positioned in front of the photomultiplier 50, thereby enabling the photomultiplier 50 to photoelectrically detect only light to be detected.

The analog data produced by photoelectrically detecting light with the photomultiplier 50 are converted by an A/D converter 53 into digital data and the digital data are fed to a data processing apparatus 54.

Although not shown in FIG. 7, the optical head 35 is constituted to be movable by a scanning mechanism in a main scanning direction indicated by an arrow X and a sub-scanning direction indicated by an arrow Y in FIG. 7 so that all of the stimulable phosphor layer regions 12 formed in the support 11 of the stimulable phosphor sheet 10 or all of the absorptive layers 4 formed in the substrate 2 of the biochemical analysis unit 1 can be scanned by the laser beam 24.

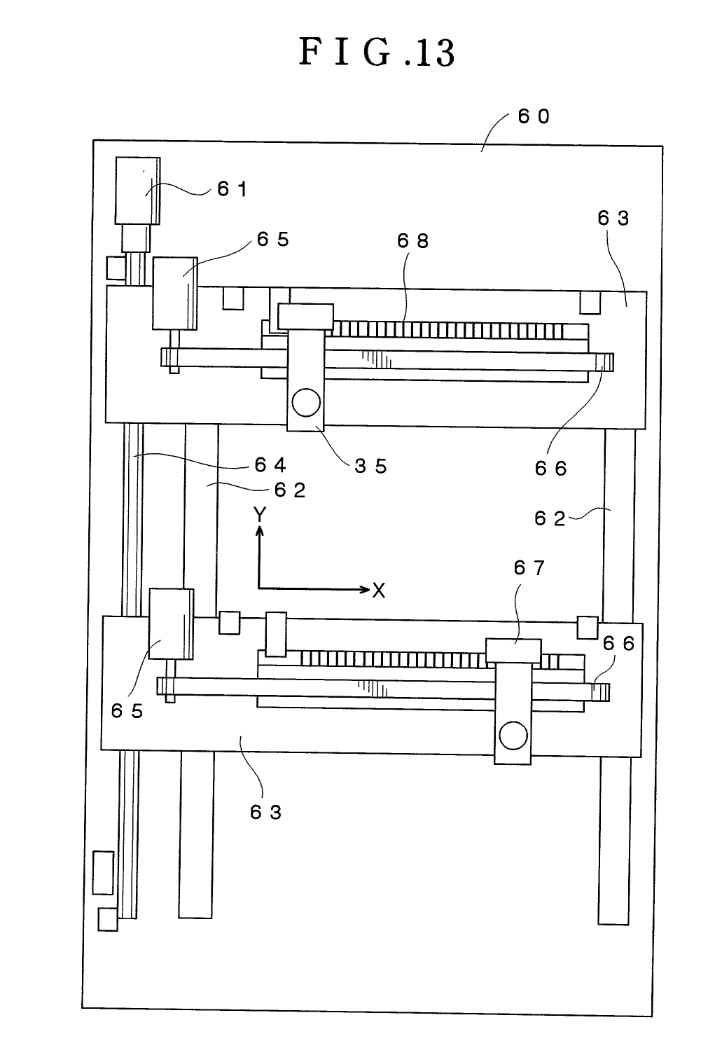

FIG. 13 is a schematic plan view showing the scanning mechanism of the optical head 35.

In FIG. 13, optical systems other than the optical head 35 and the paths of the laser beam 24 and stimulated emission 45 or fluorescence emission 45 are omitted for simplification.

As shown in FIG. 13, the scanning mechanism of the optical head 35 includes a base plate 60, and a sub-scanning pulse motor 61 and a pair of rails 62, 62 are fixed on the base plate 60. A movable base plate 63 is further provided so as to be movable in the sub-scanning direction indicated by an arrow Y in FIG. 13.

The movable base plate 63 is formed with a threaded hole (not shown) and a threaded rod 64 rotated by the sub-scanning pulse motor 61 is engaged with the inside of the hole.

A main scanning stepping motor 65 is provided on the movable base plate 63. The main scanning stepping motor 65 is adapted for intermittently driving an endless belt 66 by a pitch equal to the distance between neighboring recesses 3 formed in the biochemical analysis unit 1.

The optical head 35 is fixed to the endless belt 66 and when the endless belt 66 is driven by the main scanning stepping motor 65, the optical head 35 is moved in the main scanning direction indicated by an arrow X in FIG. 13.

In FIG. 13, the reference numeral 67 designates a linear encoder for detecting the position of the optical head 35 in the main scanning direction and the reference numeral 68 designates slits of the linear encoder 67.

Therefore, the optical head 35 is moved in the main scanning direction indicated by the arrow X and the sub-scanning direction indicated by the arrow Y in FIG. 13 by driving the endless belt 66 in the main scanning direction by the main scanning stepping motor 65 and intermittently moving the movable base plate 63 in the sub-scanning direction by the sub-scanning pulse motor 61, thereby scanning all of the stimulable phosphor layer regions 12 formed in the support 11 of the stimulable phosphor sheet 10 or all of the absorptive layers 4 formed in the substrate 2 of the biochemical analysis unit 1 with the laser beam 24.

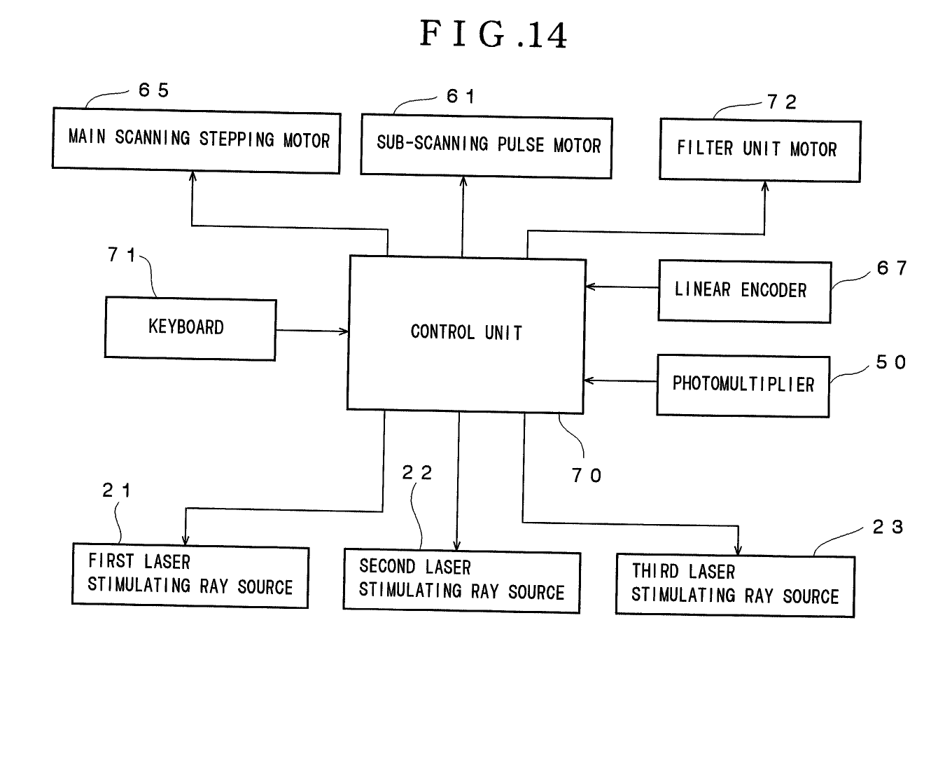

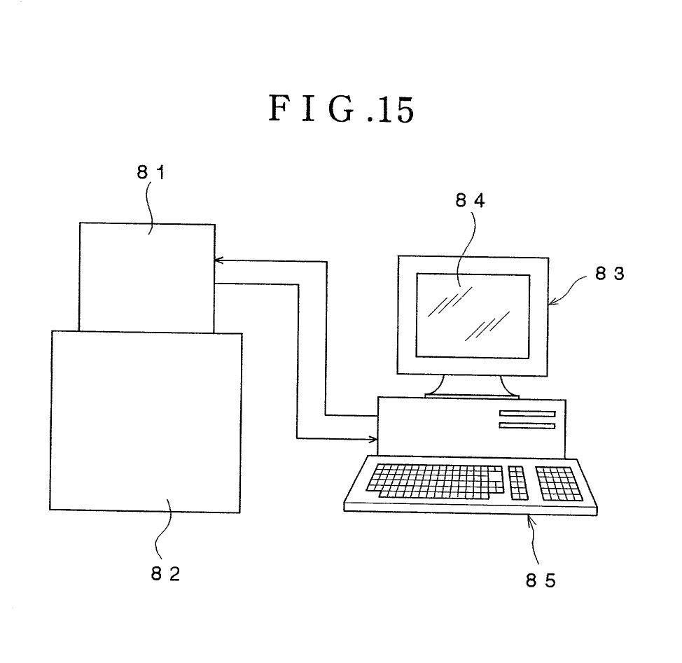

FIG. 14 is a block diagram of a control system, an input system, a drive system and a detection system of the scanner which is a preferred embodiment of the present invention.

As shown in FIG. 14, the control system of the scanner includes a control unit 70 for controlling the overall operation of the scanner, and the input system of the scanner includes a keyboard 71 which can be operated by a user and through which various instruction signals can be input.

As shown in FIG. 14, the drive system of the scanner includes the main scanning stepping motor 65 for intermittently moving the optical head 35 in the main scanning direction, the sub-scanning pulse motor 61 for intermittently moving the optical head 35 in the sub-scanning direction and a filter unit motor 72 for moving the filter unit 48 provided with the four filter members 51a, 51b, 51c and 51d.

The control unit 70 is adapted for selectively outputting a drive signal to the first laser stimulating ray source 21, the second laser stimulating ray source 22 or the third laser stimulating ray source 23 and outputting a drive signal to the filter unit motor 72.

As shown in FIG. 14, the detection system of the scanner includes the photomultiplier 50 and the linear encoder 67.

In this embodiment, the control unit 70 is adapted to control the on and off operation of the first laser stimulating ray source 21, the second laser stimulating ray source 22 or the third laser stimulating ray source 23 in accordance with a detection signal indicating the position of the optical head 35 input from the linear encoder 67.

The radiation data recorded in a number of the stimulable phosphor layer regions 12 of the stimulable phosphor sheet 10 by exposing a number of the stimulable phosphor layer regions 12 of the stimulable phosphor sheet 10 to the radioactive labeling substance contained in a number of the absorptive layers 4 formed on the inner surfaces 3a of the recesses 3 formed in the biochemical analysis unit 1 are read by the thus constituted scanner.

A stimulable phosphor sheet 10 is first set on the glass plate 41 of the stage 40 by a user.

An instruction signal indicating that the stimulable phosphor layer regions 12 formed in the support 11 of the stimulable phosphor sheet 10 are to be scanned with a laser beam 24 is then input by the user through the keyboard 71.

The instruction signal input through the keyboard is output to the control unit 70 and when the control unit 70 receives the instruction signal, it outputs a drive signal to the filter unit motor 72 in accordance with the instruction signal, thereby moving the filter unit 48 to locate the filter member 51d provided with the filter 52d having a property of transmitting only light having a wavelength corresponding to that of stimulated emission emitted from stimulable phosphor and cutting off light having a wavelength of 640 nm in the optical path of stimulated emission 45 released from the stimulable phosphor layer regions 12.

The control unit 70 further outputs a drive signal to the main scanning stepping motor 65 to move the optical head 35 in the main scanning direction and when it judges based on a detection signal indicating the position of the optical head 35 input from the linear encoder 67 that the optical head 35 has reached a position where a laser beam 24 can be projected onto a first stimulable phosphor layer region 12 among a number of the stimulable phosphor layer regions 12 formed in the support 11 of the stimulable phosphor sheet 10, it outputs a drive stop signal to the main scanning stepping motor 65 and a drive signal to the first laser stimulating ray source 21, thereby actuating it to emit a laser beam 24 having a wavelength of 640 nm.

A laser beam 24 emitted from the first laser stimulating source 21 passes through the collimator lens 25, thereby being made a parallel beam, and is reflected by the mirror 26.

The laser beam 24 reflected by the mirror 26 passes through the first dichroic mirror 27 and the second dichroic mirror 28 and advances to the mirror 29.

The laser beam 24 advancing to the mirror 29 is reflected by the mirror 29 and advances to the mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected by the concave mirror 38 and enters the optical head 35.

The laser beam 24 entering the optical head 35 is reflected by the mirror 36 and condensed by the aspherical lens 37 onto the first stimulable phosphor layer region 12 of the stimulable phosphor sheet 10 placed on the glass plate 41 of a stage 40.

In this embodiment, since the stimulable phosphor layer regions 12 are formed by embedding stimulable phosphor in the recesses 13 formed in the support 11 made of stainless steel capable of attenuating light energy, it is possible to effectively prevent the laser beam 24 from scattering in each of the stimulable phosphor layer regions 12 and entering the neighboring stimulable phosphor layer regions 12 to excite stimulable phosphor contained in the neighboring stimulable phosphor layer regions 12.

When the laser beam 24 impinges onto the first stimulable phosphor layer region 12 formed in the support 11 of the stimulable phosphor sheet 10, stimulable phosphor contained in the first stimulable phosphor layer region 12 formed in the stimulable phosphor sheet 10 is excited by the laser beam 24, thereby releasing stimulated emission 45 from the first stimulable phosphor layer region 12.

The stimulated emission 45 released from the first stimulable phosphor layer region 12 is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 advancing to the concave mirror 38 is reflected by the concave mirror 38 and advances to the perforated mirror 34.

As shown in FIG. 8, the stimulated emission 45 advancing to the perforated mirror 34 is reflected downward by the perforated mirror 34 formed as a concave mirror and advances to the filter 52d of the filter unit 48.

Since the filter 52d has a property of transmitting only light having a wavelength corresponding to that of stimulated emission emitted from stimulable phosphor and cutting off light having a wavelength of 640 nm, light having a wavelength of 640 nm corresponding to that of the stimulating ray is cut off by the filter 52d and only light having a wavelength corresponding to that of stimulated emission passes through the filter 52d to be photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When a predetermined time, for example, several microseconds, has passed after the first laser stimulating ray source 21 was turned on, the control unit 70 outputs a drive stop signal to the first laser stimulating ray source 21, thereby turning it off and outputs a drive signal to the main scanning stepping motor 65, thereby moving the optical head 35 by one pitch equal to the distance between neighboring stimulable phosphor layer regions 12 formed in the support 11 of the stimulable phosphor sheet 10.

When the control unit 70 judges based on a detection signal indicating the position of the optical head 35 input from the linear encoder 67 that the optical head 35 has been moved by one pitch equal to the distance between neighboring stimulable phosphor layer regions 12, it outputs a drive signal to the first laser stimulating ray source 21 to turn it on, thereby causing the laser beam 24 to excite stimulable phosphor contained in a second stimulable phosphor layer region 12 formed in the support 11 of the stimulable phosphor sheet 10 next to the first stimulable phosphor layer region 12.

Similarly to the above, the second stimulable phosphor layer region 12 formed in the support 11 of the stimulable phosphor sheet 10 is irradiated with the laser beam 24 for a predetermined time and when stimulated emission 45 released from the second stimulable phosphor layer region 12 is photoelectrically detected by the photomultiplier 50, the control unit 70 outputs a drive stop signal to the first laser stimulating ray source 21, thereby turning it off and outputs a drive signal to the main scanning stepping motor 65, thereby moving the optical head 35 by one pitch equal to the distance between neighboring stimulable phosphor layer regions 12.

In this manner, the on and off operation of the first laser stimulating ray source 21 is repeated in synchronism with the intermittent movement of the optical head 35 and when the control unit 70 judges based on a detection signal indicating the position of the optical head 35 input from the linear encoder 67 that the optical head 35 has been moved by one scanning line in the main scanning direction and that the stimulable phosphor layer regions 12 included in a first line of the stimulable phosphor layer regions 12 formed in the support 11 of the stimulable phosphor sheet 10 have been scanned with the laser beam 24, it outputs a drive signal to the main scanning stepping motor 65, thereby returning the optical head 35 to its original position and outputs a drive signal to the sub-scanning pulse motor 61, thereby causing it to move the movable base plate 63 by one scanning line in the sub-scanning direction.

When the control unit 70 judges based on a detection signal indicating the position of the optical head 35 input from the linear encoder 67 that the optical head 35 has been returned to its original position and judges that the movable base plate 63 has been moved by one scanning line in the sub-scanning direction, similarly to the manner in which the stimulable phosphor layer regions 12 included in the first line of the stimulable phosphor layer regions 12 formed in the support 11 of the stimulable phosphor sheet 10 were sequentially irradiated with the laser beam 24 emitted from the first laser stimulating ray source 21, the stimulable phosphor layer regions 12 included in a second line of the stimulable phosphor layer regions 12 formed in the support 11 of the stimulable phosphor sheet 10 are sequentially irradiated with the laser beam 24 emitted from the first laser stimulating ray source 21, thereby exciting stimulable phosphor contained in the stimulable phosphor layer regions 12 included in the second line and stimulated emission 45 released from the stimulable phosphor layer regions 12 is sequentially and photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When all of the stimulable phosphor layer regions 12 formed in the support 11 of the stimulable phosphor sheet 10 have been scanned with the laser beam 24 in this manner and digital data produced by photoelectrically detecting stimulated emission 45 released from stimulable phosphor contained in the stimulable phosphor layer regions 12 in response to the excitation with the laser beam 24 by the photomultiplier 50 to produce analog data and digitizing the analog data by the A/D converter 53 have been forwarded to the data processing apparatus 54, the control unit 70 outputs a drive stop signal to the first laser stimulating ray source 21, thereby turning it off.

Thus, radiation data recorded in a number of the stimulable phosphor layer regions 12 formed in the support 11 of the stimulable phosphor sheet 10 are read and biochemical analysis data are produced.

On the other hand, when fluorescence data of a fluorescent substance recorded in a number of the absorptive layers 4 formed on the inner surfaces 3a of the recesses 4 formed in the biochemical analysis unit 1 are to be read to produce biochemical analysis data, a biochemical analysis unit 1 is first set on the glass plate 41 of the stage 40 by a user.

A fluorescent substance identification signal for identifying the kind of fluorescent substance as a labeling substance is then input through the keyboard 71 by the user together with an instruction signal indicating that fluorescence data are to be read.

The fluorescent substance identification signal and the instruction signal are input to the control unit 70 and when the control unit 70 receives them, it determines the laser stimulating ray source to be used in accordance with a table stored in a memory (not shown) and also determines what filter is to be positioned in the optical path of fluorescence emission 45 among the filters 52a, 52b and 52c.

For example, when Rhodamine (registered trademark), which can be most efficiently stimulated by a laser beam having a wavelength of 532 nm, is used as a fluorescent substance for labeling a substance derived from a living organism and a signal indicating such a fact is input, the control unit 70 selects the second laser stimulating ray source 22 and the filter 52b and outputs a drive signal to the filter unit motor 72, thereby moving the filter unit 48 so that the filter member 51b inserting the filter 52b having a property of cutting off light having a wavelength of 532 nm but transmitting light having a wavelength longer than 532 nm in the optical path of the fluorescence emission 45.

The control unit 70 further outputs a drive signal to the main scanning stepping motor 65 to move the optical head 35 in the main scanning direction and when it judges based on a detection signal indicating the position of the optical head 35 input from the linear encoder 67 that the optical head 35 has reached a position where a laser beam 24 can be projected onto an absorptive layer 4 formed on the inner surface 3a of a first recess 4 formed in the biochemical analysis unit 1 among a number of the recesses 4 formed in the substrate 2 of the biochemical analysis unit 1, it outputs a drive stop signal to the main scanning stepping motor 65 to the second laser stimulating ray source 22, thereby actuating it to emit a laser beam 24 having a wavelength of 532 nm.

The laser beam 24 emitted from the second laser stimulating ray source 22 is made a parallel beam by the collimator lens 30, advances to the first dichroic mirror 27 and is reflected thereby.

The laser beam 24 reflected by the first dichroic mirror 27 transmits through the second dichroic mirror 28 and advances to the mirror 29.

The laser beam 24 advancing to the mirror 29 is reflected by the mirror 29 and further advances to the mirror 32 to be reflected thereby.

The laser beam 24 reflected by the mirror 32 advances to the perforated mirror 34 and passes through the hole 33 of the perforated mirror 34. Then, the laser beam 24 advances to the concave mirror 38.

The laser beam 24 reflected by the mirror 32 advances to the perforated mirror 34 and passes through the hole 33 of the perforated mirror 34. Then, the laser beam 24 advances to the concave mirror 38.

The laser beam 24 advancing to the concave mirror 38 is reflected thereby and enters the optical head 35.

The laser beam 24 entering the optical head 35 is reflected by the mirror 36 and condensed by the aspherical lens 37 onto an absorptive layer 4 formed in a first recess 4 of the biochemical analysis unit 1 placed on the glass plate 41 of the stage 40.

In this embodiment, since the substrate 2 of the biochemical analysis unit 1 is made of aluminum capable of attenuating light energy, it is possible to effectively prevent the laser beam 24 from scattering in the substrate 2 of the biochemical analysis unit 1 and entering the neighboring recesses 4.

When the laser beam 24 enters the absorptive region 4 formed in the first recess 4 of the biochemical analysis unit 1, a fluorescent substance such as a fluorescent dye, for instance, Rhodamine, contained in the absorptive layer 4 formed in the first recess 3 of the biochemical analysis unit 1 is stimulated by the laser beam 24 and fluorescence emission 45 is released from the Rhodamine.

In this embodiment, since the substrate 2 of the biochemical analysis unit 1 is made of aluminum capable of attenuating light energy, it is possible to reliably prevent fluorescence emission released from a fluorescent substance from being mixed with fluorescent released from a fluorescent substance contained in the neighboring absorptive layer 4.

The fluorescence emission 45 released from Rhodamine is condensed by the aspherical lens 37 provided in the optical head 35 and reflected by the mirror 36 on the side of an optical path of the laser beam 24, thereby being made a parallel beam to advance to the concave mirror 38.

The fluorescence emission 45 advancing to the concave mirror 38 is reflected by the concave mirror 38 and advances to the perforated mirror 34.

As shown in FIG. 8, the fluorescence emission 45 advancing to the perforated mirror 34 is reflected downward by the perforated mirror 34 formed as a concave mirror and advances to the filter 52b of a filter unit 48.

Since the filter 52b has a property of cutting off light having a wavelength of 532 nm but transmitting light having a wavelength longer than 532 nm, light having the same wavelength of 532 nm as that of the stimulating ray is cut off by the filter 52b and only light in the wavelength of the fluorescence emission 45 released from Rhodamine passes through the filter 52b to be photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When a predetermined time, for example, several microseconds, has passed after the second laser stimulating ray source 22 was turned on, the control unit 70 outputs a drive stop signal to the second laser stimulating ray source 22, thereby turning it off and outputs a drive signal to the main scanning stepping motor 65, thereby moving the optical head 35 by one pitch equal to the distance between neighboring recesses 3 formed in the biochemical analysis unit 1 in the main scanning direction.

When the control unit 70 judges based on a detection signal indicating the position of the optical head 35 input from the linear encoder 67 that the optical head 35 has been moved by one pitch equal to the distance between neighboring recesses 3 in the main scanning direction and the optical head 35 has reached a position where a laser beam 24 can be projected onto an absorptive layer 4 formed on the inner surface 3a of a second recess 3 formed in the biochemical analysis unit 1, it outputs a drive stop signal to the main scanning stepping motor 65 and a drive signal to the second laser stimulating ray source 22, thereby turning it on and exciting a fluorescent substance, for example, Rhodamine, contained in the absorptive layer 4 formed on the inner surface 3a of the second recess 3 formed in the biochemical analysis unit 1 with the laser beam 24.

Similarly to the above, the absorptive layer 4 formed on the inner surface 3a of the second recess 3 formed in the biochemical analysis unit 1 is irradiated with the laser beam 24 for a predetermined time and when fluorescence emission 45 released from the absorptive layer 4 formed on the inner surface 3a of the second recess 3 is photoelectrically detected by the photomultiplier 50 to produce analog data, the control unit 70 outputs a drive stop signal to the second laser stimulating ray source 22, thereby turning it off and outputs a drive signal to the main scanning stepping motor 65, thereby moving the optical head 35 by one pitch equal to the distance between neighboring recesses 3 of the biochemical analysis unit 1.

In this manner, the on and off operation of the second laser stimulating ray source 22 is repeated in synchronism with the intermittent movement of the optical head 35 and when the control unit 70 judges based on a detection signal indicating the position of the optical head 35 input from the linear encoder 67 that the optical head 35 has been moved by one scanning line in the main scanning direction and that the absorptive layers 4 formed on the inner surfaces 3a of all of the recesses 3 included in a first line of the recesses 3 formed in the substrate 2 of the biochemical analysis unit 1 have been scanned with the laser beam 24, it outputs a drive signal to the main scanning stepping motor 65, thereby returning the optical head 35 to its original position and outputs a drive signal to the sub-scanning pulse motor 61, thereby causing it to move the movable base plate 63 by one scanning line in the sub-scanning direction.

When the control unit 70 judges based on a detection signal indicating the position of the optical head 35 input from the linear encoder 67 that the optical head 35 has been returned to its original position and judges that the movable base plate 63 has been moved by one scanning line in the sub-scanning direction, similarly to the manner in which the absorptive layers 4 formed on the inner surfaces 3a of the recesses 3 included in the first line of the recesses 3 formed in the substrate 2 of the biochemical analysis unit 1 were sequentially irradiated with the laser beam 24 emitted from the second laser stimulating ray source 22, the absorptive layers 4 formed on the inner surfaces 3a of the recesses 3 included in a second line of the recesses 3 formed in the substrate 2 of the biochemical analysis unit 1 are sequentially irradiated with the laser beam 24 emitted from the second laser stimulating ray source 22, thereby exciting Rhodamine contained in the absorptive layers 4 formed on the inner surfaces 3a of the recesses 3 included in the second line and fluorescence emission 45 released from the absorptive layers 4 is sequentially and photoelectrically detected by the photomultiplier 50.

Analog data produced by photoelectrically detecting fluorescence emission by the photomultiplier 50 are converted by the A/D converter 53 to digital data to be forwarded to the data processing apparatus 54.

When all of the absorptive layers 4 formed on the inner surfaces 3a of the recesses 4 formed in the substrate 2 of the biochemical analysis unit 1 have been scanned with the laser beam 24 in this manner and digital data produced by photoelectrically detecting fluorescence emission 45 released from the absorptive layers 4 by the photomultiplier 50 to analog data and digitizing the analog data by the A/D converter 53 have been forwarded to the data processing apparatus 54, the control unit 70 outputs a drive stop signal to the second laser stimulating ray source 22m thereby turning it off.

Thus, fluorescence data recorded in the absorptive layers 4 formed on the inner surfaces 3a of a number of the recesses 3 formed in the substrate 2 of the biochemical analysis unit 1 are read and biochemical analysis data are produced.

FIG. 15 is a schematic front view showing a data producing system for reading chemiluminescence data of a labeling substance recorded in the absorptive layers 4 formed on the inner surfaces 3a of a number of the recesses formed in the biochemical analysis unit 1, which generates chemiluminescence emission when it contacts a chemiluminescent substrate and producing biochemical analysis data.

The data producing system shown in FIG. 15 is constituted to be able to also read fluorescence data of a fluorescent substance such as a fluorescent dye recorded in the absorptive layers 4 formed on the inner surfaces 3a of a number of the recesses formed in the biochemical analysis unit 1.

As shown in FIG. 15, the data producing system includes a cooled CCD camera 81, a dark box 82 and a personal computer 83. As shown in FIG. 15, the personal computer 83 is equipped with a CRT 84 and a keyboard 85.

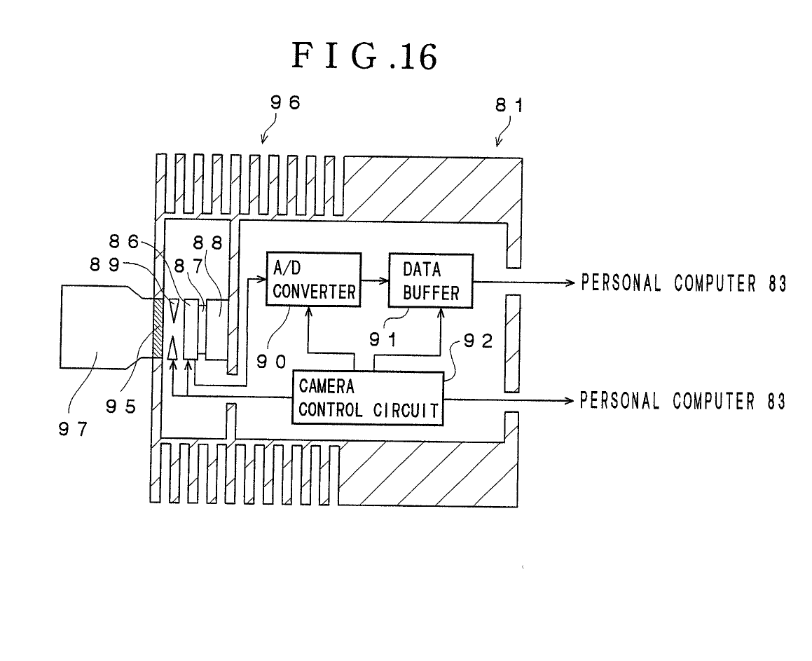

FIG. 16 is a schematic longitudinal cross sectional view showing the cooled CCD camera 81.

As shown in FIG. 15, the cooled CCD camera 81 includes a CCD 86, a heat transfer plate 87 made of metal such as aluminum, a Peltier element 88 for cooling the CCD 86, a shutter 89 disposed in front of the CCD 86, an A/D converter 90 for converting analog data produced by the CCD 86 to digital data, a data buffer 91 for temporarily storing the data digitized by the A/D converter 90, and a camera control circuit 92 for controlling the operation of the cooled CCD camera 81. An opening formed between the dark box 82 and the cooled CCD camera 81 is closed by a glass plate 95 and the periphery of the cooled CCD camera 81 is formed with heat dispersion fins 96 over substantially its entire length for dispersing heat.

A camera lens 97 disposed in the dark box 82 is mounted on the front surface of the glass plate 95 disposed in the cooled CCD camera 81.

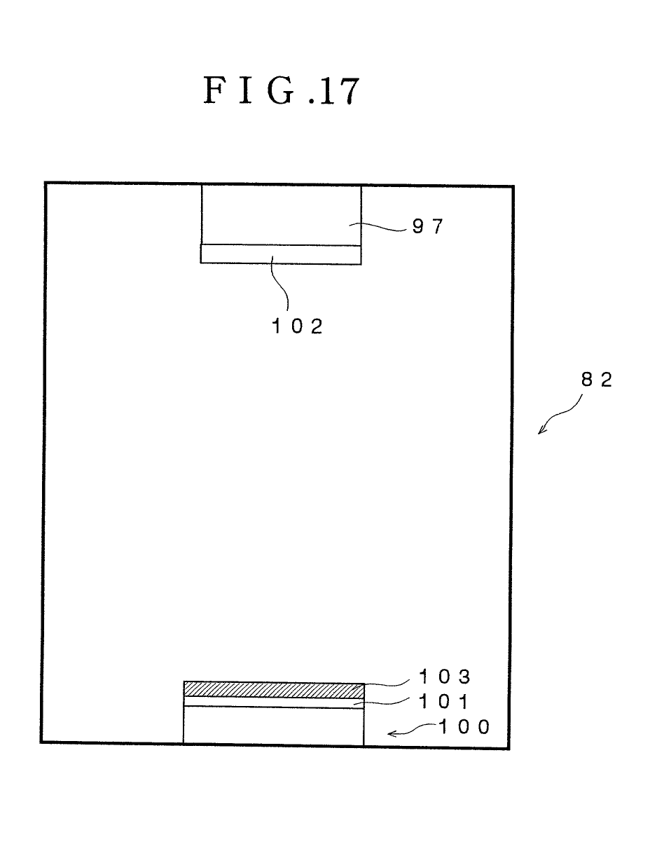

FIG. 17 is a schematic vertical cross sectional view showing the dark box 82.

As shown in FIG. 17, the dark box 82 is equipped with a light emitting diode stimulating ray source 100 for emitting a stimulating ray. The light emitting diode stimulating ray source 100 is provided with a filter 101 detachably mounted thereon and a diffusion plate 102 mounted on the upper surface of the filter 101. The stimulating ray is emitted via the diffusion plate 102 toward a biochemical analysis unit (not shown) placed on the diffusion plate 102 so as to ensure that the biochemical analysis unit can be uniformly irradiated with the stimulating ray. The filter 101 has a property of cutting light components having a wavelength not close to that of the stimulating ray and harmful to the stimulation of a fluorescent substance and transmitting through only light components having a wavelength in the vicinity of that of the stimulating ray. A filter 102 for cutting light components having a wavelength in the vicinity of that of the stimulating ray is detachably provided on the front surface of the camera lens 97.

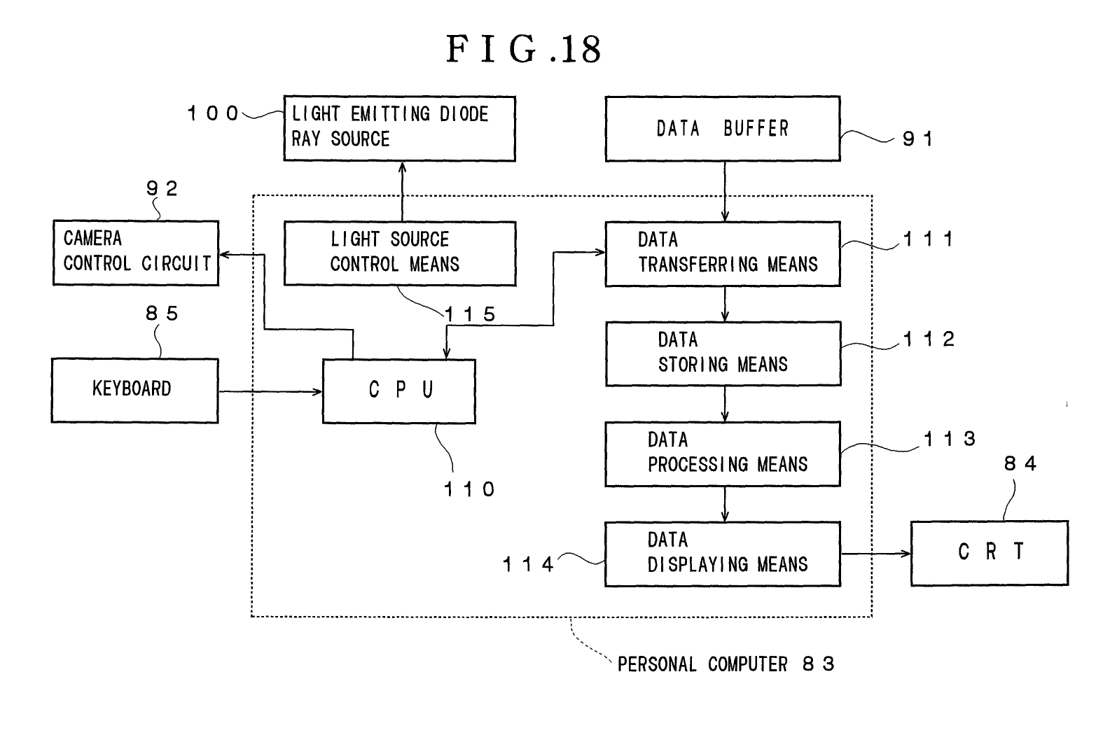

FIG. 18 is a block diagram of the personal computer 83 and peripheral devices thereof.

As shown in FIG. 18, the personal computer 83 includes a CPU 110 for controlling the exposure of the cooled CCD camera 81, a data transferring means 111 for reading the data produced by the cooled CCD camera 81 from the data buffer 91, a storing means 112 for storing data, a data processing means 113 for effecting data processing on the digital data stored in the data storing means 112, and a data displaying means 114 for displaying visual data on the screen of the CRT 84 based on the digital data stored in the data storing means 112. The light emitting diode stimulating ray source 100 is controlled by a light source control means 115 and an instruction signal can be input via the CPU 110 to the light source control means 115 through the keyboard 85. The CPU 110 is constituted so as to output various signals to the camera controlling circuit 92 of the cooled CCD camera 81.

The data producing system shown in FIGS. 14 to 17 is constituted so as to detect chemiluminescence emission generated by the contact of a labeling substance contained in the absorptive layers 4 formed on the inner surfaces 3a of a number of the recesses 3 formed in the biochemical analysis unit 1 and a chemiluminescent substrate, with the CCD 86 of the cooled CCD camera 81 through a camera lens 97, thereby reading chemiluminescence data to produce biochemical analysis data, and irradiate the biochemical analysis unit 1 with a stimulating ray emitted from the light emitting diode stimulating ray source 100 and detect fluorescence emission released from a fluorescent substance such as a fluorescent dye contained in the absorptive layers 4 formed on the inner surfaces 3a of a number of the recesses 3 formed in the biochemical analysis unit 1 upon being stimulated, with the CCD 86 of the cooled CCD camera 81 through a camera lens 97, thereby reading fluorescence data to produce biochemical analysis data.

When biochemical analysis data are to be produced by reading chemiluminescence data, the filter 102 is removed and while the light emitting diode stimulating ray source 100 is kept off, the biochemical analysis unit 1 is placed on the diffusion plate 103, which is releasing chemiluminescence emission as a result of contact of a labeling substance contained in the absorptive layers 4 formed on the inner surfaces 3a of a number of the recesses 3 formed in the biochemical analysis unit 1 and a chemiluminescent substrate. The lens focus is then adjusted by the user using the camera lens 97 and the dark box 92 is closed.

When an exposure start signal is input by the user through the keyboard 85, the exposure start signal is input through the CPU 110 to the camera control circuit 92 of the cooled CCD camera 81 so that the shutter 88 is opened by the camera control circuit 92, whereby the exposure of the CCD 86 is started.

Chemiluminescence emission released from a number of the absorptive layers 4 of the biochemical analysis unit 1 impinges on the light receiving surface of the CCD 86 of the cooled CCD camera 81 via the camera lens 97, thereby forming an image on the light receiving surface. The CCD 86 receives light of the thus formed image and accumulates it in the form of electric charges therein.

In this embodiment, since the substrate 2 of the biochemical analysis unit 1 is made of aluminum, it is possible to reliably prevent chemiluminescence emission released from the labeling substance contained in each of the absorptive layers 4 from scattering in the substrate 2 and being mixed with chemiluminescence emission released from a labeling substance contained in absorptive layers 4 formed on the inner surfaces 3a of the neighboring recesses 3.

When a predetermined exposure time has passed, the CPU 110 outputs an exposure completion signal to the camera control circuit 92 of the cooled CCD camera 81.

When the camera controlling circuit 92 receives the exposure completion signal from the CPU 110, it transfers analog data accumulated in the CCD 86 in the form of electric charge to the A/D converter 90 to cause the AID converter 90 to digitize the data and to temporarily store the thus digitized data in the data buffer 91.

At the same time, the CPU 110 outputs a data transfer signal to the data transferring means 111 to cause it to read out the digital data from the data buffer 91 of the cooled CCD camera 81 and to input them to the data storing means 112.

When the user inputs a data producing signal through the keyboard 85, the CPU 110 outputs the digital data stored in the data storing means 112 to the data processing means 113 and causes the data processing means 113 to effect data processing on the digital data in accordance with the user's instructions. The CPU 110 then outputs a data display signal to the displaying means 115 and causes the displaying means 115 to display biochemical analysis data on the screen of the CRT 84 based on the thus processed digital data.

On the other hand, when biochemical analysis data are to be produced by reading fluorescence data, the biochemical analysis unit 1 is first placed on the diffusion plate 103.

The light emitting diode stimulating ray source 100 is then turned on by the user and the lens focus is adjusted using the camera lens 97. The dark box 92 is then closed.

When the user inputs an exposure start signal through the keyboard 85, the light emitting diode stimulating ray source 100 is again turned on by the light source control means 115, thereby emitting a stimulating ray toward the biochemical analysis unit 1.

At the same time, the exposure start signal is input via the CPU 110 to the camera control circuit 92 of the cooled CCD camera 81 and the shutter 89 is opened by the camera control circuit 92, whereby the exposure of the CCD 86 is started.

The stimulating ray emitted from the light emitting diode stimulating ray source 100 passes through the filter 101, whereby light components of wavelengths not in the vicinity of that of the stimulating ray are cut. The stimulating ray then passes through the diffusion plate 103 to be made uniform light and the biochemical analysis unit 1 is irradiated with the uniform stimulating ray.

When the biochemical analysis unit 1 is irradiated with the stimulating ray, a fluorescent substance such as a fluorescent dye contained in the absorptive layer 4 formed on the inner surfaces 3a of a number of the recesses 3 of the biochemical analysis unit 1 is stimulated by the stimulating ray, thereby releasing fluorescence emission from a number of the absorptive layers 4.

The fluorescence emission released from a number of the absorptive layers 4 the biochemical analysis unit 1 impinges on the light receiving surface of the CCD 86 of the cooled CCD camera 81 through the filter 102 and the camera lens 97 and forms an image thereon. The CCD 86 receives light of the thus formed image and accumulates it in the form of electric charges therein. Since light components of wavelength equal to the stimulating ray wavelength are cut by the filter 102, only fluorescence emission released from the fluorescent substance contained in the absorptive layers 4 formed on the inner surfaces 3a of a number of the absorptive regions 4 of the biochemical analysis unit 1 is received by the CCD 86.

In this embodiment, since the substrate 2 of the biochemical analysis unit 1 is made of aluminum capable of attenuating light energy, it is possible to reliably prevent fluorescence emission released from the fluorescent substance such as a fluorescent dye from scattering in the substrate 2 and being mixed with fluorescence emission released from a fluorescent substance such as a fluorescent dye contained in the absorptive layers 4 formed on the surfaces of the neighboring recesses 3.

When a predetermined exposure time has passed, the CPU 110 outputs an exposure completion signal to the camera control circuit 92 of the cooled CCD camera 81.

When the camera controlling circuit 92 receives the exposure completion signal from the CPU 110, it transfers analog data accumulated in the CCD 86 in the form of electric charge to the AID converter 90 to cause the A/D converter 90 to digitize the data and to temporarily store the thus digitized data in the data buffer 91.

At the same time, the CPU 110 outputs a data transfer signal to the data transferring means 111 to cause it to read out the digital data from the data buffer 91 of the cooled CCD camera 81 and to input them to the data storing means 112.

When the user inputs a data producing signal through the keyboard 85, the CPU 110 outputs the digital data stored in the data storing means 112 to the data processing apparatus 113 and causes the data processing apparatus 113 to effect data processing on the digital data in accordance with the user's instructions. The CPU 110 then outputs a data display signal to the displaying means 115 and causes the displaying means 115 to display biochemical analysis data on the screen of the CRT 84 based on the thus processed digital data.

In this embodiment, chemiluminescence data recorded in a number of the absorptive layers 4 formed in the biochemical analysis unit 1 can be transferred onto a stimulable phosphor sheet and biochemical analysis data can be produced by reading chemiluminescence data transferred onto the stimulable phosphor sheet by a scanner described later.

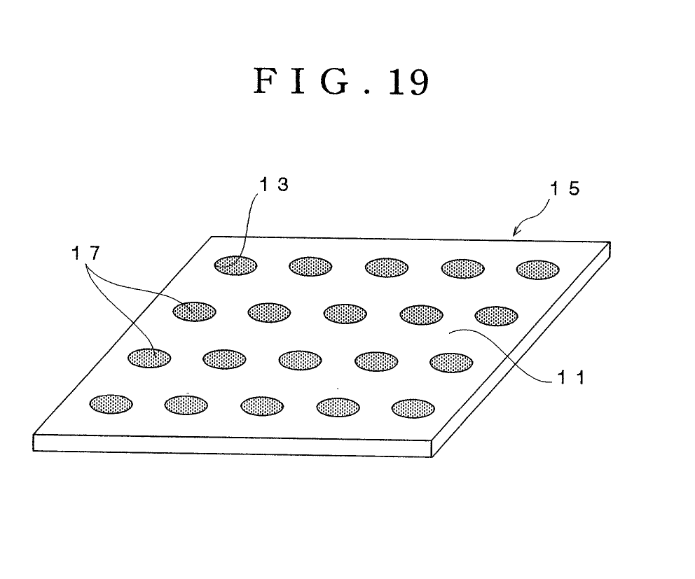

FIG. 19 is a schematic perspective view showing a stimulable phosphor sheet onto which chemiluminescence data recorded in a number of the absorptive layers 4 of the biochemical analysis unit 1 are to be transferred.

A stimulable phosphor sheet 15 shown in FIG. 19 has the same configuration as that of the stimulable phosphor sheet 10 shown in FIG. 5 except that a number of stimulable phosphor layer regions 17 are formed by embedding SrS system stimulable phosphor capable of absorbing and storing light energy in the recesses 13 formed in the support 11.

Chemiluminescence data recorded in a number of the absorptive layers 4 formed in the biochemical analysis unit 1 are transferred onto a number of the stimulable phosphor layer regions 17 of the stimulable phosphor 15 shown in FIG. 19.

When chemiluminescence data recorded in a number of the absorptive layers 4 formed in the biochemical analysis unit 1 are to be transferred onto a number of the stimulable phosphor layer regions 17 of the stimulable phosphor 15, a number of the absorptive layers 4 of the biochemical analysis unit 1 are first brought into contact with a chemiluminescent substrate.

As a result, chemiluminescence emission in a wavelength of visible light is selectively released from a number of the absorptive layers 4 of the biochemical analysis unit 1.

Similarly to the manner shown in FIG. 6, the stimulable phosphor sheet 15 is then superposed on the biochemical analysis unit 1 formed of a number of the absorptive layers 4 selectively releasing chemiluminescence emission in such a manner that each of the stimulable phosphor layer regions 17 formed in the support 11 of the stimulable phosphor sheet 15 face the corresponding recess 3 formed in the biochemical analysis unit 1.

In this manner, each of the stimulable phosphor layer regions 17 formed in the stimulable phosphor sheet 15 is kept to face the corresponding absorptive layer 4 formed in the biochemical analysis unit 1 for a predetermined time period, whereby a number of the stimulable phosphor layer regions 17 formed in the stimulable phosphor sheet 15 are exposed to chemiluminescence emission selectively released from a number of the absorptive regions 4 formed in the biochemical analysis unit 1.

In this embodiment, since the substrate 2 of the biochemical analysis unit 1 is made of aluminum capable of attenuating light energy, chemiluminescence emission released from the absorptive layers 4 of the biochemical analysis unit 1 can be efficiently prevented from scattering in the substrate 2 of the biochemical analysis unit 1. Further, since the support 11 of the stimulable phosphor sheet 15 is made of stainless steel capable of attenuating light energy, chemiluminescence emission released from the absorptive layers 4 of the biochemical analysis unit 1 can be efficiently prevented from scattering in the support 11 of the stimulable phosphor sheet 15. Therefore, it is possible to reliably prevent the chemiluminescence emission released from each of the absorptive layers 4 of the biochemical analysis unit 1 from entering the stimulable phosphor layer regions 17 which the recesses 3 next to the recess 3 in which the absorptive layer 4 is formed face.

In this manner, chemiluminescence data are transferred onto and recorded in a number of the stimulable phosphor layer regions 17 formed in the support 11 of the stimulable phosphor sheet 15.

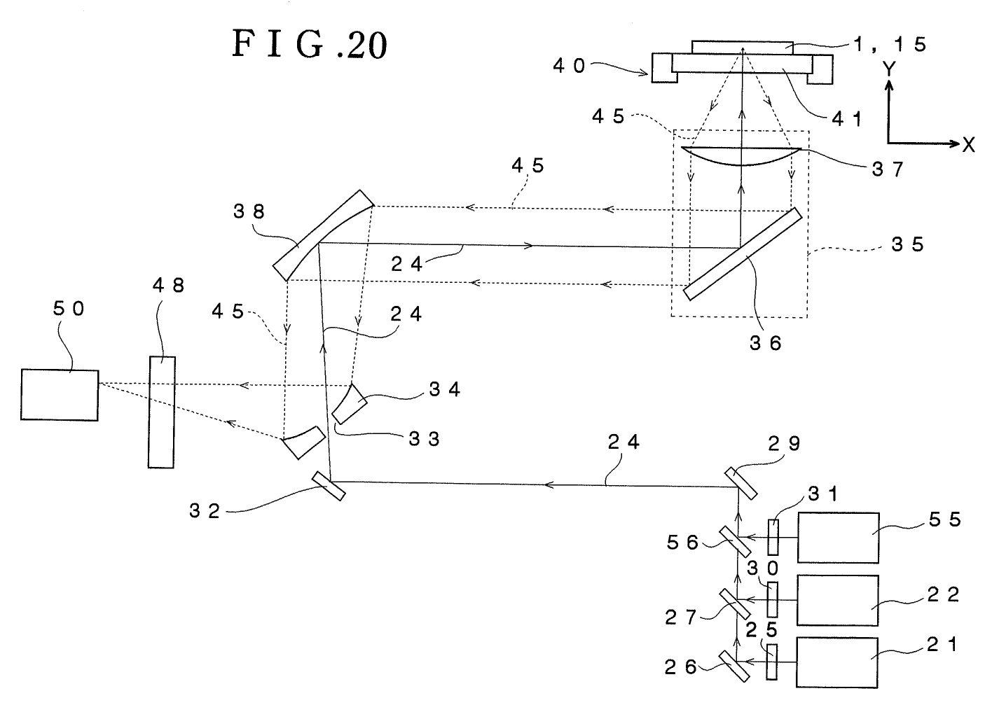

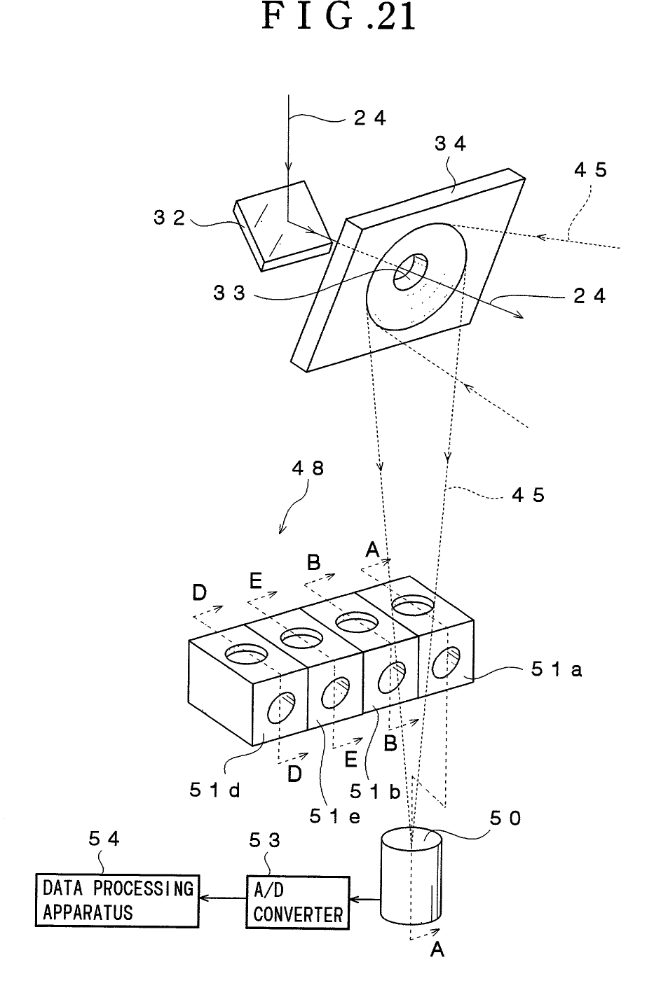

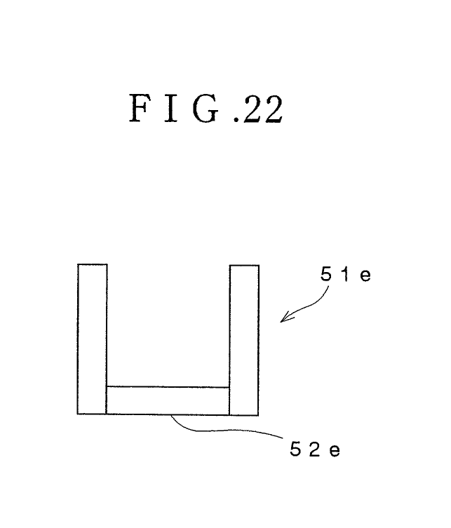

FIG. 20 is a schematic perspective view showing a scanner for reading chemiluminescence data recorded in a number of the stimulable phosphor layer regions 17 formed in the support 11 of the stimulable phosphor sheet 15 and producing biochemical analysis data, FIG. 21 is a schematic perspective view showing details in the vicinity of a photomultiplier of a scanner shown in FIG. 20 and FIG. 22 is a schematic cross-sectional view taken along a line E-E in FIG. 21.

A scanner shown in FIGS. 20 to 22 has the same configuration as that of the scanner shown in FIGS. 7 to 14 except that it includes a fourth laser stimulating ray source 55 for emitting a laser beam 24 having a wavelength of 980 nm which can effectively stimulate SrS system stimulable phosphor instead of the third laser stimulating ray source 23 for emitting a laser beam 24 having a wavelength of 473 nm, includes a filter member 51e provided with a filter having a property of transmitting only light having a wavelength corresponding to that of stimulated emission emitted from stimulable phosphor and cutting off light having a wavelength of 980 nm, and includes a third dichroic mirror 56 for transmitting light having a wavelength equal to and shorter than 640 nm but reflecting light having a wavelength of 980 nm instead of the second dichroic mirror 28 for transmitting light having a wavelength equal to and longer than 532 nm but reflecting light having a wavelength of 473 nm.

The thus constituted scanner reads chemiluminescence data recorded in a number of the stimulable phosphor layer regions 17 of the stimulable phosphor sheet 15 and produces biochemical analysis data in the following manner.

A stimulable phosphor sheet 15 is first set on the glass plate 41 of the stage 40 by a user.

An instruction signal indicating that radiation data recorded in the stimulable phosphor layer regions 17 formed in the stimulable phosphor sheet 15 are to be read is then input through the keyboard 71.

The instruction signal input through the keyboard 71 is input to the control unit 70 and the control unit 70 outputs a drive signal to the filter unit motor 72 in accordance with the instruction signal, thereby moving the filter unit 48 so as to locate the filter member 51e provided with a filter 52e having a property of transmitting only light having a wavelength corresponding to that of stimulated emission emitted from the stimulable phosphor layer regions 17 and cutting off light having a wavelength of 980 nm in the optical path of stimulated emission 45.

The control unit 70 further outputs a drive signal to the main scanning stepping motor 65 to move the optical head 35 in the main scanning direction and when it determines based on a detection signal indicating the position of the optical head 35 input from the linear encoder 67 that the optical head 35 has reached a position where a laser beam 24 can be projected onto a first stimulable phosphor layer region 17 among a number of the stimulable phosphor layer regions 17 formed in the stimulable phosphor sheet 15, it outputs a drive stop signal to the main scanning stepping motor 65 and a drive signal to the fourth stimulating ray source 55, thereby actuating it to emit a laser beam 24 having a wavelength of 980 nm.

A laser beam 24 emitted from the fourth laser stimulating ray source 23 passes through a collimator lens 31, thereby being made a parallel beam, and is reflected by the third dichroic mirror 56, thereby changing its direction by 90 degrees. The laser beam 24 then advances to the mirror 29.

The laser beam 24 advancing to the mirror 29 is reflected by the mirror 29 and advances to the mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected by the concave mirror 38 and enters the optical head 35.

The laser beam 24 entering the optical head 35 is reflected by the mirror 36 and condensed by the aspherical lens 37 onto the first stimulable phosphor layer region 17 of the stimulable phosphor sheet 15 placed on the glass plate 41 of a stage 40.

In this embodiment, since the stimulable phosphor layer regions 17 are formed by embedding stimulable phosphor in the recesses 13 formed in the support 11 made of stainless steel capable of attenuating light energy, it is possible to effectively prevent the laser beam 24 from scattering in each of the stimulable phosphor layer regions 17 and entering the neighboring stimulable phosphor layer regions 17 to excite stimulable phosphor contained in the neighboring stimulable phosphor layer regions 17.

When the laser beam 24 impinges onto the first stimulable phosphor layer region 17 formed in the stimulable phosphor sheet 15, stimulable phosphor contained in the first stimulable phosphor layer region 17 formed in the stimulable phosphor sheet 15 is excited by the laser beam 24, thereby releasing stimulated emission 45 from the first stimulable phosphor layer region 17.

The stimulated emission 45 released from the first stimulable phosphor layer region 17 of the stimulable phosphor sheet 15 is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 advancing to the concave mirror 38 is reflected by the concave mirror 38 and advances to the perforated mirror 34.

As shown in FIG. 21, the stimulated emission 45 advancing to the perforated mirror 34 is reflected downward by the perforated mirror 34 formed as a concave mirror and advances to the filter 52e of the filter unit 48.

Since the filter 52e has a property of transmitting only light having a wavelength corresponding to that of stimulated emission emitted from stimulable phosphor and cutting off light having a wavelength of 980 nm, light having a wavelength of 980 nm corresponding to that of the stimulating ray is cut off by the filter 52e and only light having a wavelength corresponding to that of stimulated emission passes through the filter 52e to be photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When a predetermined time, for example, several microseconds, has passed after the fourth stimulating ray source 55 was turned on, the control unit 70 outputs a drive stop signal to the fourth stimulating ray source 55, thereby turning it off and outputs a drive signal to the main scanning stepping motor 65, thereby moving the optical head 35 by one pitch equal to the distance between neighboring stimulable phosphor layer regions 17 of the stimulable phosphor sheet 15.

When the control unit 70 determines based on a detection signal indicating the position of the optical head 35 input from the linear encoder 67 that the optical head 35 has been moved by one pitch equal to the distance between neighboring stimulable phosphor layer regions 17, it outputs a drive signal to the fourth stimulating ray source 55 to turn it on, thereby causing the laser beam 24 to excite stimulable phosphor contained in a second stimulable phosphor layer region 17 formed in the support 11 of the stimulable phosphor sheet 15 next to the first stimulable phosphor layer region 17.

Similarly to the above, the second stimulable phosphor layer region 17 formed in the stimulable phosphor sheet 15 is irradiated with the laser beam 24 for a predetermined time and when stimulated emission 45 released from the second stimulable phosphor layer region 17 is photoelectrically detected by the photomultiplier 50, the control unit 70 outputs a drive stop signal to the fourth stimulating ray source 55, thereby turning it off and outputs a drive signal to the main scanning stepping motor 65, thereby moving the optical head 35 by one pitch equal to the distance between neighboring stimulable phosphor layer regions 17.

In this manner, the on and off operation of the fourth stimulating ray source 55 is repeated in synchronism with the intermittent movement of the optical head 35 and when the control unit 70 determines based on a detection signal indicating the position of the optical head 35 input from the linear encoder 67 that the optical head 35 has been moved by one scanning line in the main scanning direction and that the stimulable phosphor layer regions 17 included in a first line of the stimulable phosphor layer regions 17 formed in the stimulable phosphor sheet 15 have been scanned with the laser beam 24, it outputs a drive signal to the main scanning stepping motor 65, thereby returning the optical head 35 to its original position and outputs a drive signal to the sub-scanning pulse motor 61, thereby causing it to move the movable base plate 63 by one scanning line in the sub-scanning direction.

When the control unit 70 determines based on a detection signal indicating the position of the optical head 35 input from the linear encoder 67 that the optical head 35 has been returned to its original position and determines that the movable base plate 63 has been moved by one scanning line in the sub-scanning direction, similarly to the manner in which the stimulable phosphor layer regions 17 included in the first line of the stimulable phosphor layer regions 17 formed in the stimulable phosphor sheet 15 were sequentially irradiated with the laser beam 24 emitted from the fourth laser stimulating ray source 55, the stimulable phosphor layer regions 17 included in a second line of the stimulable phosphor layer regions 17 formed in the stimulable phosphor sheet 15 are sequentially irradiated with the laser beam 24 emitted from the fourth laser stimulating ray source 55, thereby exciting stimulable phosphor contained in the stimulable phosphor layer regions 17 included in the second line and stimulated emission 45 released from the stimulable phosphor layer regions 17 is sequentially and photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When all of the stimulable phosphor layer regions 17 formed in the support 11 of the stimulable phosphor sheet 15 have been scanned with the laser beam 24 released from the fourth laser stimulating ray source 55 to excite stimulable phosphor contained in the stimulable phosphor layer regions 17 and biochemical analysis data produced from radiation data recorded in the stimulable phosphor layer regions 17 formed in the support 11 of the stimulable phosphor sheet 15 by photoelectrically detecting stimulated emission 45 released from the stimulable phosphor layer regions 17 with the photomultiplier 50 to produce analog data and digitizing the analog data by the A/D converter 53 have been forwarded to the data processing apparatus 54, the control unit 70 outputs a drive stop signal to the fourth laser stimulating ray source 55, thereby turning it off.

As described above, chemiluminescence data recorded in a number of the stimulable phosphor layer regions 17 of the stimulable phosphor sheet 15 are read by the scanner to produce biochemical analysis data.

When the production of biochemical analysis data has been completed, the biochemical analysis unit 1 is washed.

In this embodiment, specific binding substances are absorbed in the absorptive layers 4 formed on the inner surfaces 3a of a number of the recesses 3 formed in the substrate 2 of the biochemical analysis unit 1 and a substance derived from a living organism and labeled with the labeling substances is hybridized with the specific binding substances contained in the absorptive layers 4. Therefore, when the biochemical analysis unit 1 is to be washed, it is sufficient to wash only the absorptive layers 4 formed on the inner surfaces 3a of the number of the recesses 3 and since each of the absorptive layers 4 has a large surface area, it is possible to efficiently wash the biochemical analysis unit for reuse.

According to this embodiment, since the substrate 2 of the biochemical analysis unit 1 is made of aluminum capable of attenuating radiation energy and light energy, when a number of the stimulable phosphor layer regions 12 formed in the support 11 of the stimulable phosphor sheet 10 are to be exposed to a radioactive labeling substance selectively contained in the absorptive layers 4 formed on the inner surfaces 3a of a number of the recesses 3 formed in the substrate 2 of the biochemical analysis unit 1, electron beams (β rays) released from the radioactive labeling substance can be effectively prevented from scattering in the substrate 2 of the biochemical analysis unit 1. Further, since each of the stimulable phosphor layer regions 12 formed in the support 11 of the stimulable phosphor sheet 10 has the same diameter as that of the corresponding recess 3 formed in the biochemical analysis unit 1 and the stimulable phosphor sheet 10 is superposed on the biochemical analysis unit 1 in such a manner that each of the stimulable phosphor layer regions 12 accurately faces the corresponding recess 3 formed in the biochemical analysis unit 1, thereby exposing a number of the stimulable phosphor layer regions 12 to the radioactive labeling substance, electron beams (β rays) released from the radioactive labeling substance contained in each of the absorptive layers 4 can be effectively prevented from entering the stimulable phosphor layer regions 12 the absorptive layers 4 formed on the inner surface 3a of the neighboring recesses 3 face. Therefore, even when the recesses 3 are formed in the substrate 2 of the biochemical analysis unit 1 at high density, it is possible to expose a number of the stimulable phosphor layer regions 12 formed in the support 11 of the stimulable phosphor sheet 10 to only radioactive labeling substance contained in the absorptive layer 4 formed on the inner surface 3a of the corresponding recess 3.

Further, according to this embodiment, since the substrate 2 of the biochemical analysis unit 1 is made of aluminum capable of attenuating radiation energy and light energy, it is possible to effectively prevent the laser beam 24 from scattering in the substrate 2 of the biochemical analysis unit 1 and stimulating a fluorescent substance such as a fluorescent dye contained in the absorptive layer 4 formed on the inner surface 3a of the neighboring recesses 4 and, therefore, it is possible to prevent noise caused by the scattering of the laser beam 24 from being generated in a biochemical analysis data produced by photoelectrically detecting fluorescence emission 45 and to improve the quantitative characteristics of biochemical analysis data.

Furthermore, according to this embodiment, since the substrate 2 of the biochemical analysis unit 1 is made of aluminum capable of attenuating radiation energy and light energy, it is possible to effectively prevent fluorescence emission released from a fluorescent substance such as a fluorescent dye in response to the stimulation with the laser beam 24 or the stimulating ray emitted from the stimulating ray source 100 from scattering in the substrate 2 of the biochemical analysis unit 1. Therefore, since mixing of fluorescence emission released from the absorptive layers 4 formed on the inner surfaces 3a of neighboring recesses 3 is prevented, even when the recesses 3 are formed in the substrate 2 of the biochemical analysis unit 1 with high density, it is possible to prevent noise caused by the scattering of fluorescence emission from being generated in biochemical analysis data produced by photoelectrically detecting fluorescence emission 45 and to improve the quantitative characteristics of biochemical analysis data.

Moreover, according to this embodiment, since the substrate 2 of the biochemical analysis unit 1 is made of aluminum capable of attenuating radiation energy and light energy, it is possible to effectively prevent chemiluminescence emission released by the contact of the labeling substance and the chemiluminescent substrate from scattering in the substrate 2 of the biochemical analysis unit 1. Therefore, since mixing of chemiluminescence emission released from the absorptive layers 4 formed on the inner surfaces 3a of neighboring recesses 3 is prevented, even when the recesses 3 are formed in the substrate 2 of the biochemical analysis unit 1 with high density, it is possible to prevent noise caused by the scattering of chemiluminescence emission from being generated in a biochemical analysis data produced by photoelectrically detecting chemiluminescence emission and to improve the quantitative characteristics of biochemical analysis data.

Further, according to this embodiment, since the substrate 2 of the biochemical analysis unit 1 is made of aluminum capable of attenuating radiation energy and light energy, when a number of the stimulable phosphor layer regions 17 formed in the support 11 of the stimulable phosphor sheet 15 are to be exposed to chemiluminescence emission selectively released from the absorptive layers 4 formed on the inner surfaces 3a of a number of the recesses 3 formed in the substrate 2 of the biochemical analysis unit 1, chemiluminescence emission selectively released from the absorptive layers 4 formed on the inner surfaces 3a of a number of the recesses 3 formed in the substrate 2 of the biochemical analysis unit 1 can be effectively prevented from scattering in the substrate 2 of the biochemical analysis unit 1. Further, since each of the stimulable phosphor layer regions 17 formed in the support 11 of the stimulable phosphor sheet 15 has the same diameter as that of the corresponding recess 3 formed in the biochemical analysis unit 1 and the stimulable phosphor sheet 15 is superposed on the biochemical analysis unit 1 in such a manner that each of the stimulable phosphor layer regions 17 accurately faces the corresponding recess 3 formed in the biochemical analysis unit 1, thereby exposing a number of the stimulable phosphor layer regions 17 to the chemiluminescence emission, chemiluminescence emission released from each of the absorptive layers 4 can be effectively prevented from entering the stimulable phosphor layer regions 17 the absorptive layers 4 formed on the inner surface 3a of the neighboring recesses 3 face. Therefore, even when the recesses 3 are formed in the substrate 2 of the biochemical analysis unit 1 at high density, it is possible to expose a number of the stimulable phosphor layer regions 17 formed in the support 11 of the stimulable phosphor sheet 15 to only chemiluminescence emission released from the absorptive layer 4 formed on the inner surface 3a of the corresponding recess 3.

Furthermore, according to this embodiment, since specific binding substances are absorbed as a probe in the absorptive layers 4 formed on the inner surfaces 3a of the individual recesses 3 and are absorbed in a region having a smaller volume than that of the case of charging a porous material into the recess 3, the rate of hybridization reaction can be improved. Further, since the hybridization reaction solution 9 can be brought into contact with the absorptive layers 3 having a large surface area, it is possible to increase the probability of a substance derived from a living organism as a target associating with the specific binding substances absorbed in the absorptive layers 4 as a probe. Therefore, the efficiency of hybridization can be markedly improved.

Moreover, according to this embodiment, specific binding substances are absorbed in the absorptive layers 4 formed on the inner surfaces 3a of a number of the recesses 3 formed in the substrate 2 of the biochemical analysis unit 1 and a substance derived from a living organism and labeled with the labeling substances is hybridized with the specific binding substances contained in the absorptive layers 4. Therefore, when the biochemical analysis unit 1 is to be washed, it is sufficient to wash only the absorptive layers 4 formed on the inner surfaces 3a of a number of the recesses 3 and since each of the absorptive layers 4 has a large surface area, it is possible to efficiently wash the biochemical analysis unit for reuse.

Furthermore, according to this embodiment, since the substrate 2 of the biochemical analysis unit 1 is made of aluminum, the biochemical analysis unit 1 does not stretch or shrink when it is subjected to liquid processing such as hybridization and, therefore, it is possible to easily and accurately superpose the stimulable phosphor sheet 10, 15 on the biochemical analysis unit 1 so that each of the stimulable phosphor layer regions 12, 17 formed in the support 11 of the stimulable phosphor sheet 10, 15 accurately faces the corresponding recess 3 formed with the absorptive layer 4 on the inner surface 3a of thereof of the biochemical analysis unit 1, thereby exposing the stimulable phosphor layer regions 12, 17.

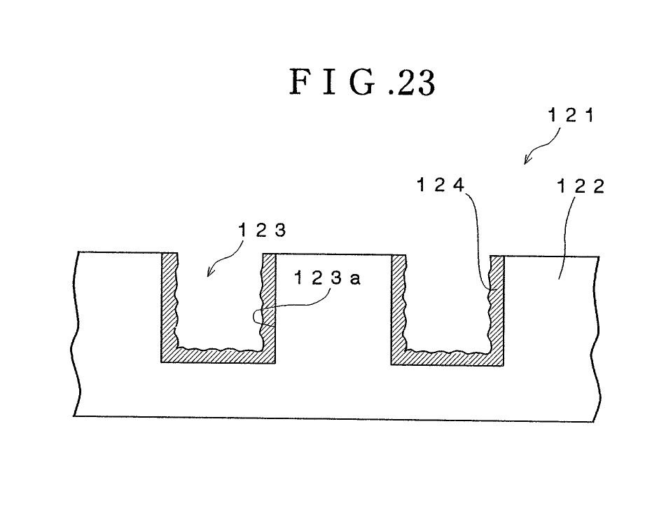

FIG. 23 is a schematic partial cross sectional view showing a biochemical analysis unit which is another preferred aspect of the present invention.

As shown in FIG. 23, in this embodiment, a biochemical analysis unit 121 includes a substrate 121 made of aluminum capable of attenuating radiation energy and light energy and having flexibility and formed with a number of substantially circular recesses 123 at high density.

As shown in FIG. 23, an absorptive layer 124 is formed of nylon-6 capable of forming a membrane filter on the inner surface 123a of each of the recesses 123. In this embodiment, the surface of each of the absorptive layers 124 is roughened so as to have a fractal structure, thereby increasing the surface area thereof.

Although not accurately shown in FIG. 23, in this embodiment, about 10,000 recesses 123 having a size of about 0.01 mm² are regularly formed at a density of about 5,000 per cm² in the substrate 122.

Similarly to the embodiment shown in FIGS. 1 and 2, in this embodiment, as shown in FIG. 3, a solution containing specific binding substances such as a plurality of cDNAs is spotted onto the absorptive layers 124 formed on the inner surfaces 123a of a number of the recesses 123.

In the biochemical analysis unit 121 according to this embodiment, since the surface of each of the absorptive layers 123 is processed so as to have a fractal structure, thereby increasing the surface area thereof, a sufficient amount of specific binding substances can be absorbed in the absorptive layer 124 formed on the inner surface 123a of each of a number of the recesses 123.

Further, as shown in FIG. 4, the biochemical analysis unit 121 is set in the hybridization reaction vessel 8 accommodating a hybridization reaction solution 9 containing a substance derived from a living organism and labeled with a radioactive labeling substance. a substance derived from a living organism and labeled with a fluorescent substance such as a fluorescent dye and a substance derived from a living organism and labeled with a labeling substance which generates chemiluminescence emission when it contacts a chemiluminescent substrate. As a result, specific binding substances absorbed in the absorptive layers 124 formed on the inner surface 123a of a number of the recesses 123 can be selectively hybridized with a substance derived from a living organism, labeled with a radioactive labeling substance and contained in the hybridization reaction solution 9, a substance derived from a living organism, labeled with a fluorescent substance such as a fluorescent dye and contained in the hybridization reaction solution 9, and a substance derived from a living organism, labeled with a labeling substance which generates chemiluminescence emission when it contacts a chemiluminescent substrate and contained in the hybridization reaction solution 9.

Thus, radiation data, fluorescence data and chemiluminescence data are recorded in the biochemical analysis unit 121.

Similarly to the previous embodiment, fluorescence data recorded in the biochemical analysis unit 121 are read by the scanner shown in FIGS. 7 to 14 or the cooled CCD camera 81 of the data producing system shown in FIGS. 15 to 18, thereby producing biochemical analysis data.

On the other hand, radiation data of the radioactive labeling substance recorded in the biochemical analysis unit 121 are transferred onto a number of the stimulable phosphor layer regions 12 formed in the support 11 of the stimulable phosphor sheet 10.

More specifically, similarly to FIG. 6, the stimulable phosphor sheet 10 is superposed on the biochemical analysis unit 121 in such a manner that a number of the stimulable phosphor layer regions 12 formed in the support 11 of the stimulable phosphor sheet 10 face the corresponding recesses 123 formed in the substrate 122 of the biochemical analysis unit 121, thereby exposing a number of the stimulable phosphor layer regions 12 to the radioactive labeling substance selectively contained in the absorptive layers 124 formed on the inner surfaces 123a of a number of the recesses 123.

Similarly to the previous embodiment, the radiation data thus transferred onto a number of the stimulable phosphor layer regions 12 formed in the support 11 of the stimulable phosphor sheet 10 are read by the scanner shown in FIGS. 7 to 14, thereby producing biochemical analysis data.

On the other hand, similarly to the previous embodiment, chemiluminescence data recorded in the biochemical analysis unit 121 are read by the cooled CCD camera 81 of the data producing system shown in FIGS. 15 to 18, thereby producing biochemical analysis data, or are transferred onto a number of the stimulable phosphor layer regions 17 of the stimulable phosphor sheet 15 shown in FIG. 19.

Similarly to the previous embodiment, chemiluminescence data transferred onto a number of the stimulable phosphor layer regions 17 of the stimulable phosphor sheet 15 are read by the scanner shown in FIGS. 20 to 22, thereby producing biochemical analysis data.

According to this embodiment, since the substrate 122 of the biochemical analysis unit 121 is made of aluminum capable of attenuating radiation energy and light energy, when a number of the stimulable phosphor layer regions 12 formed in the support 11 of the stimulable phosphor sheet 10 are to be exposed to a radioactive labeling substance selectively contained in the absorptive layers 124 formed on the inner surfaces 123a of a number of the recesses 123 formed in the substrate 122 of the biochemical analysis unit 121, electron beams (β rays) released from the radioactive labeling substance contained in the absorptive layers 124 formed on the inner surfaces 123a of a number of the recesses 123 can be effectively prevented from scattering in the substrate 122 of the biochemical analysis unit 121. Further, since the stimulable phosphor sheet 10 is superposed on the biochemical analysis unit 121 in such a manner that each of the stimulable phosphor layer regions 12 formed in the support 11 of the stimulable phosphor sheet 10 accurately faces the corresponding recess 123 formed in the substrate 122 of the biochemical analysis unit 121, electron beams (β rays) released from the radioactive labeling substance contained in the absorptive layer 124 formed on the inner surface 123a of each of the recesses 123 formed in the biochemical analysis unit 121 enter only the corresponding stimulable phosphor layer region 12, thereby enabling each of the stimulable phosphor layer regions 12 to be selectively exposed to only the radioactive labeling substance contained in the absorptive layer 124 formed on the inner surface 123a of the corresponding recess 123 formed in the biochemical analysis unit 121. Therefore, it is possible to prevent noise caused by exposing the stimulable phosphor layer region 12 to be exposed to the radioactive labeling substance contained in the absorptive layer 124 formed on the inner surface 123a of the corresponding recess 123 of the biochemical analysis unit 121 to electron beams (β rays) released from the radioactive labeling substance contained in the absorptive layers 124 formed on the inner surfaces 123a of the neighboring recesses 123 from being generated in biochemical analysis data and the quantitative characteristics of biochemical analysis data can be improved.

Further, according to this embodiment, since the substrate 122 of the biochemical analysis unit 121 is made of aluminum capable of attenuating radiation energy and light energy, when a number of the stimulable phosphor layer regions 17 formed in the support 11 of the stimulable phosphor sheet 15 are to be exposed to chemiluminescence emission selectively released from the absorptive layers 124 formed on the inner surfaces 123a of a number of the recesses 123 formed in the substrate 122 of the biochemical analysis unit 121, chemiluminescence emission selectively released from the absorptive layers 124 formed on the inner surfaces 123a of a number of the recesses 123 formed in the substrate 122 of the biochemical analysis unit 121 can be effectively prevented from scattering in the substrate 122 of the biochemical analysis unit 121. Further, since the stimulable phosphor sheet 15 is superposed on the biochemical analysis unit 121 in such a manner that each of the stimulable phosphor layer regions 17 accurately faces the corresponding recess 123 formed in the biochemical analysis unit 121, chemiluminescence emission released from the absorptive layer 124 formed on the inner surface 123a of each of the recesses 123 formed in the biochemical analysis unit 121 enter only the corresponding stimulable phosphor layer region 17, thereby enabling each of the stimulable phosphor layer regions 17 to be selectively exposed to only the chemiluminescence emission released from the absorptive layer 124 formed on the inner surface 123a of the corresponding recess 123 formed in the biochemical analysis unit 121. Therefore, it is possible to prevent noise caused by exposing the stimulable phosphor layer region 17 to be exposed to the chemiluminescence emission released from the absorptive layer 124 formed on the inner surface 123a of the corresponding recess 123 of the biochemical analysis unit 121 to chemiluminescence emission released from the absorptive layers 124 formed on the inner surfaces 123a of the neighboring recesses 123 from being generated in biochemical analysis data and the quantitative characteristics of biochemical analysis data can be improved.

Furthermore, according to this embodiment, since specific binding substances are absorbed as a probe in the absorptive layers 124 formed on the inner surfaces 123a of the individual recesses 123 and are absorbed in a region having a smaller volume than that of the case of charging a porous material into the recess 123, the rate of hybridization reaction can be improved. Further, since the hybridization reaction solution 9 can be brought into contact with the absorptive layers 123 having a large surface area, it is possible to increase the probability of a substance derived from a living organism as a target associating with the specific binding substances absorbed in the absorptive layers 124 as a probe. Therefore, the efficiency of hybridization can be markedly improved.

Moreover, according to this embodiment, specific binding substances are absorbed in the absorptive layers 124 formed on the inner surfaces 123a of a number of the recesses 123 formed in the substrate 122 of the biochemical analysis unit 121 and a substance derived from a living organism and labeled with the labeling substances is hybridized with the specific binding substances contained in the absorptive layers 124. Therefore, when the biochemical analysis unit 121 is to be washed, it is sufficient to wash only the absorptive layers 124 formed on the inner surfaces 123a of a number of the recesses 123 and since each of the absorptive layers 124 has a large surface area, it is possible to efficiently wash the biochemical analysis unit for reuse.

Further, according to this embodiment, since each of the absorptive layers is processed so as to have a fractal structure and the surface area thereof is increased, a sufficient amount of the specific binding substance can be absorbed in each of the absorptive layers 124.

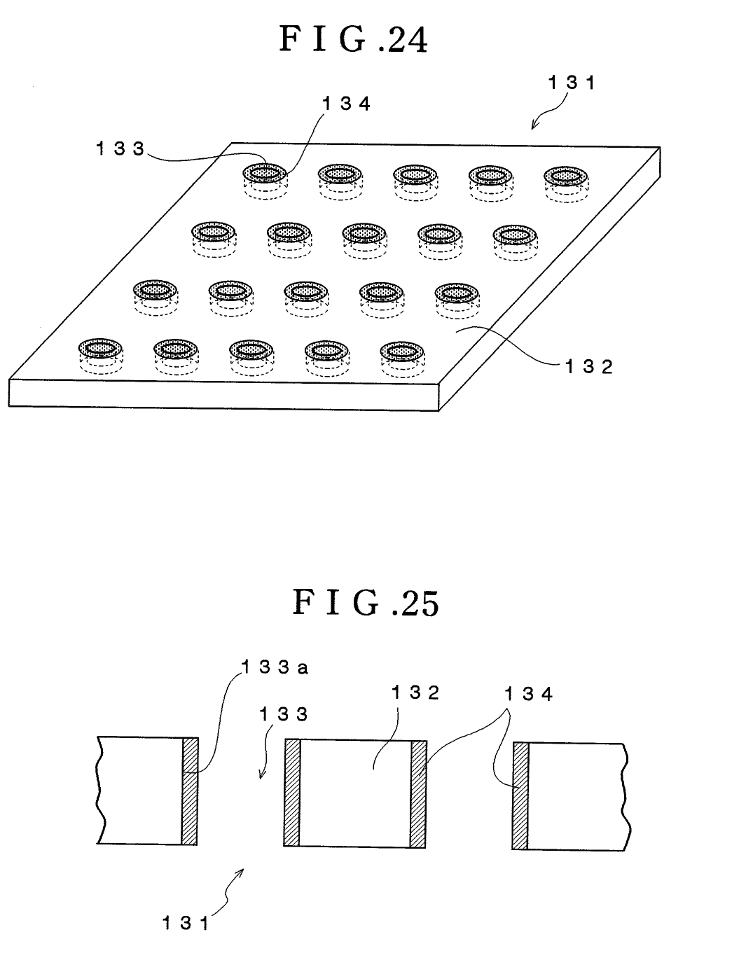

FIG. 24 is a schematic perspective view showing a biochemical analysis unit which is a further preferred embodiment of the present invention and FIG. 25 is a schematic partial cross sectional view thereof.

As shown in FIGS. 24 and 25, a biochemical analysis unit 131 according to this embodiment includes a substrate 132 made of aluminum capable of attenuating radiation energy and light energy and having flexibility and formed with a number of substantially circular through-holes 133 at high density.

As shown in FIG. 24, an absorptive layer 134 is formed of nitrocellulose capable of forming a membrane filter on the inner surface 133a of each of the through-holes 133.

Although not accurately shown in FIG. 24, in this embodiment, about 10,000 recesses 133 having a size of about 0.01 mm² are regularly formed at a density of about 5,000 per cm² in the substrate 132.

Similarly to the embodiment shown in FIGS. 1 and 2, in this embodiment, as shown in FIG. 3, a solution containing specific binding substances such as a plurality of cDNAs is spotted onto the absorptive layers 134 formed on the inner surfaces 133a of a number of the recesses 133. As shown in FIG. 4, the biochemical analysis unit 131 is then set in the hybridization reaction vessel 8 accommodating a hybridization reaction solution 9 containing a substance derived from a living organism and labeled with a radioactive labeling substance. a substance derived from a living organism and labeled with a fluorescent substance such as a fluorescent dye and a substance derived from a living organism and labeled with a labeling substance which generates chemiluminescence emission when it contacts a chemiluminescent substrate. As a result, specific binding substances absorbed in the absorptive layers 134 formed on the inner surface 133a of a number of the recesses 133 can be selectively hybridized with a substance derived from a living organism, labeled with a radioactive labeling substance and contained in the hybridization reaction solution 9, a substance derived from a living organism, labeled with a fluorescent substance such as a fluorescent dye and contained in the hybridization reaction solution 9, and a substance derived from a living organism, labeled with a labeling substance which generates chemiluminescence emission when it contacts a chemiluminescent substrate and contained in the hybridization reaction solution 9.

Thus, radiation data, fluorescence data and chemiluminescence data are recorded in the biochemical analysis unit 131.

Similarly to the previous embodiment, fluorescence data recorded in the biochemical analysis unit 131 are read by the scanner shown in FIGS. 7 to 14 or the cooled CCD camera 81 of the data producing system shown in FIGS. 15 to 18, thereby producing biochemical analysis data.

On the other hand, radiation data of the radioactive labeling substance recorded in the biochemical analysis unit 131 are transferred onto a number of the stimulable phosphor layer regions 12 formed in the support 11 of the stimulable phosphor sheet 10.

More specifically, similarly to FIG. 6, the stimulable phosphor sheet 10 is superposed on the biochemical analysis unit 131 in such a manner that a number of the stimulable phosphor layer regions 12 formed in the support 11 of the stimulable phosphor sheet 10 face the corresponding recesses 133 formed in the substrate 132 of the biochemical analysis unit 131, thereby exposing a number of the stimulable phosphor layer regions 12 to the radioactive labeling substance selectively contained in the absorptive layers 134 formed on the inner surfaces 133a of a number of the recesses 133.

Similarly to the previous embodiment, the radiation data thus transferred onto a number of the stimulable phosphor layer regions 12 formed in the support 11 of the stimulable phosphor sheet 10 are read by the scanner shown in FIGS. 7 to 14, thereby producing biochemical analysis data.

On the other hand, similarly to the previous embodiment, chemiluminescence data recorded in the biochemical analysis unit 131 are read by the cooled CCD camera 81 of the data producing system shown in FIGS. 15 to 18, thereby producing biochemical analysis data, or transferred onto a number of the stimulable phosphor layer regions 17 of the stimulable phosphor sheet 15.

Similarly to the previous embodiment, chemiluminescence data transferred onto a number of the stimulable phosphor layer regions 17 of the stimulable phosphor sheet 15 are read by the scanner shown in FIGS. 20 to 22, thereby producing biochemical analysis data.

According to this embodiment, since the substrate 132 of the biochemical analysis unit 131 is made of aluminum capable of attenuating radiation energy and light energy, when a number of the stimulable phosphor layer regions 12 formed in the support 11 of the stimulable phosphor sheet 10 are to be exposed to a radioactive labeling substance selectively contained in the absorptive layers 134 formed on the inner surfaces 133a of a number of the through-holes 133 formed in the substrate 132 of the biochemical analysis unit 131, electron beams (β rays) released from the radioactive labeling substance contained in the absorptive layers 134 formed on the inner surfaces 133a of a number of the through-holes 133 can be effectively prevented from scattering in the substrate 132 of the biochemical analysis unit 131. Further, since the stimulable phosphor sheet 10 is superposed on the biochemical analysis unit 131 in such a manner that each of the stimulable phosphor layer regions 12 formed in the support 11 of the stimulable phosphor sheet 10 accurately faces the corresponding through-hole 133 formed in the substrate 132 of the biochemical analysis unit 131, electron beams (β rays) released from the radioactive labeling substance contained in the absorptive layer 134 formed on the inner surface 133a of each of the through-holes 133 formed in the biochemical analysis unit 131 enter only the corresponding stimulable phosphor layer region 12, thereby enabling each of the stimulable phosphor layer regions 12 to be selectively exposed to only the radioactive labeling substance contained in the absorptive layer 134 formed on the inner surface 133a of the corresponding through-hole 133 formed in the biochemical analysis unit 131. Therefore, it is possible to prevent noise caused by exposing the stimulable phosphor layer region 12 to the radioactive labeling substance contained in the absorptive layer 134 formed on the inner surface 133a of the corresponding through-hole 133 of the biochemical analysis unit 131 to electron beams (β rays) released from the radioactive labeling substance contained in the absorptive layers 134 formed on the inner surfaces 133a of the neighboring through-holes 133 from being generated in biochemical analysis data and the quantitative characteristics of biochemical analysis data can be improved.

Further, according to this embodiment, since the substrate 132 of the biochemical analysis unit 131 is made of aluminum capable of attenuating radiation energy and light energy, when a number of the stimulable phosphor layer regions 17 formed in the support 11 of the stimulable phosphor sheet 15 are to be exposed to chemiluminescence emission selectively released from the absorptive layers 134 formed on the inner surfaces 133a of a number of the through-holes 133 formed in the substrate 132 of the biochemical analysis unit 131, chemiluminescence emission selectively released from the absorptive layers 134 formed on the inner surfaces 133a of a number of the through-holes 133 formed in the substrate 132 of the biochemical analysis unit 131 can be effectively prevented from scattering in the substrate 132 of the biochemical analysis unit 131. Further, since the stimulable phosphor sheet 15 is superposed on the biochemical analysis unit 131 in such a manner that each of the stimulable phosphor layer regions 17 accurately faces the corresponding through-holes 133 formed in the biochemical analysis unit 131, chemiluminescence emission released from the absorptive layer 134 formed on the inner surface 133a of each of the through-holes 133 formed in the biochemical analysis unit 131 enter only the corresponding stimulable phosphor layer region 17, thereby enabling each of the stimulable phosphor layer regions 17 to be selectively exposed to only the chemiluminescence emission released from the absorptive layer 134 formed on the inner surface 133a of the corresponding through-hole 133 formed in the biochemical analysis unit 131. Therefore, it is possible to prevent noise caused by exposing the stimulable phosphor layer region 17 to be exposed to the chemiluminescence emission released from the absorptive layer 134 formed on the inner surface 133a of the corresponding through-hole 133 of the biochemical analysis unit 131 to chemiluminescence emission released from the absorptive layers 134 formed on the inner surfaces 133a of the neighboring through-holes 133 from being generated in biochemical analysis data and the quantitative characteristics of biochemical analysis data can be improved.

Furthermore, according to this embodiment, since specific binding substances are absorbed as a probe in the absorptive layers 134 formed on the inner surfaces 133a of the individual through-holes 133 and are absorbed in a region having a smaller volume than that of the case of charging a porous material into the through-hole 133, the rate of hybridization reaction can be improved. Further, since the hybridization reaction solution 9 can be brought into contact with the absorptive layers 134 having a large surface area, it is possible to increase the probability of a substance derived from a living organism as a target associating with the specific binding substances absorbed in the absorptive layers 134 as a probe. Therefore, the efficiency of hybridization can be markedly improved.

Moreover, according to this embodiment, specific binding substances are absorbed in the absorptive layers 134 formed on the inner surfaces 133a of a number of the through-holes 133 formed in the substrate 132 of the biochemical analysis unit 131 and a substance derived from a living organism and labeled with the labeling substances is hybridized with the specific binding substances contained in the absorptive layers 134. Therefore, when the biochemical analysis unit 131 is to be washed, it is sufficient to wash only the absorptive layers 134 formed on the inner surfaces 133a of a number of the through-holes 133 and since each of the absorptive layers 134 has a large surface area, it is possible to efficiently wash the biochemical analysis unit for reuse.

The present invention has thus been shown and described with reference to specific embodiments. However, it should be noted that the present invention is in no way limited to the details of the described arrangements but changes and modifications may be made without departing from the scope of the appended claims.

For example, in the above described embodiments, as specific binding substances, cDNAs each of which has a known base sequence and is different from the others are used. However, specific binding substances usable in the present invention are not limited to cDNAs but all specific binding substances capable of specifically binding with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, can be employed in the present invention as a specific binding substance.

Further, in the above described embodiments, although the substrate 2, 122, 132 of the biochemical analysis unit 1, 121, 131 is made of aluminum, it is not absolutely necessary to make the substrate 2, 122, 132 of the biochemical analysis unit 1, 121, 131 of aluminum and insofar as the substrate 2, 122, 132 of the biochemical analysis unit 1, 121, 131 is made of material capable of attenuating radiation energy and/or light energy, the material for forming the substrate 2, 122, 132 of the biochemical analysis unit 1, 121, 131 is not particularly limited. The substrate 2, 122, 132 of the biochemical analysis unit 1, 121, 131 can be formed of either inorganic compound material or organic compound material and is preferably formed of metal material, ceramic material or plastic material. Illustrative examples of inorganic compound materials usable for forming the substrate 2, 122, 132 of the biochemical analysis unit 1 include metals such as gold, silver, copper, zinc, aluminum, titanium, tantalum, chromium, steel, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. High molecular compounds are preferably used as organic compound material and illustrative examples thereof include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4,10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Furthermore, in the above described embodiments, although the substrate 2, 122, 132 of the biochemical analysis unit 1, 121, 131 is made of aluminum capable of attenuating radiation energy and light energy, in the case where biochemical analysis data are produced by reading only radiation data recorded in a number of stimulable phosphor layer regions 12 formed in the support 11 of the stimulable phosphor sheet 10, the substrate 2, 122, 132 of the biochemical analysis unit 1, 121, 131 may be made of a material having a property of transmitting light energy but attenuating radiation energy. On the other hand, in the case where biochemical analysis data are produced by reading only chemiluminescence data or fluorescence data recorded in the absorptive layers 4, 124, 134 formed in the substrate 2, 122, 132 of the biochemical analysis unit 1, 121, 131, the substrate 2, 122, 132 of the biochemical analysis unit 1, 121, 131 may be made of a material having a property of transmitting radiation energy but attenuating light energy and it is not absolutely necessary to make the substrate 2, 122, 132 of the biochemical analysis unit 1, 121, 131 of a material capable of attenuating both radiation energy and light energy.

Moreover, although about 10,000 substantially circular recesses 3, 123 having a size of about 0.01 mm² are regularly formed at a density of about 5,000 per cm² in the substrate 2, 122 in the embodiment shown in FIGS. 1 and 2 and the embodiment shown in FIG. 23 and about 10,000 substantially circular through-holes 133 having a size of about 0.01 mm² are regularly formed at a density of about 5,000 per cm² in the substrate 132 in the embodiment shown in FIGS. 24 and 25, the shape of each of the recesses 2, 122 or the through-holes 133 is not limited to substantially a circular shape but may be formed in an arbitrary shape, for example, a rectangular shape.

Further, although about 10,000 substantially circular recesses 3, 123 having a size of about 0.01 mm² are regularly formed at a density of about 5,000 per cm² in the substrate 2, 122 in the embodiment shown in FIGS. 1 and 2 and the embodiment shown in FIG. 23 and about 10,000 substantially circular through-holes 133 having a size of about 0.01 mm² are regularly formed at a density of about 5,000 per cm² in the substrate 132 in the embodiment shown in FIGS. 24 and 25, the number or size of the recesses 3, 123 or the through-holes 133 may be arbitrarily selected in accordance with the purpose. Preferably, 10 or more of the recesses 3, 123 or the through-holes 133 having a size of 5 mm² or less are formed in the substrate 2, 122, 132 of the biochemical analysis unit 1, 120, 130 at a density of 10/cm² or less.

Furthermore, although about 10,000 substantially circular recesses 3, 123 having a size of about 0.01 mm² are regularly formed at a density of about 5,000 per cm in the substrate 2, 122 in the embodiment shown in FIGS. 1 and 2 and the embodiment shown in FIG. 23 and about 10,000 substantially circular through-holes 133 having a size of about 0.01 mm² are regularly formed at a density of about 5,000 per cm² in the substrate 132 in the embodiment shown in FIGS. 24 and 25, it is not absolutely necessary to regularly form the recesses 3, 123 or the through-holes 133 in the substrate 2, 122, 132 of the biochemical analysis unit 1.

Moreover, in the above described embodiments, a hybridization reaction solution 9 containing a substance derived from a living organism and labeled with a radioactive labeling substance, a substance derived from a living organism and labeled with a fluorescent substance such as a fluorescent dye, and a substance derived from a living organism and labeled with a labeling substance which generates chemiluminescence emission when it contacts a chemiluminescent substrate is prepared and the substance derived from a living organism and labeled with a radioactive labeling substance, the substance derived from a living organism and labeled with a fluorescent substance such as a fluorescent dye, and the substance derived from a living organism and labeled with a labeling substance which generates chemiluminescence emission when it contacts a chemiluminescent substrate is selectively hybridized with the specific binding substances contained in a number of the absorptive layers 3, 124, 134 of the biochemical analysis unit 1, 121, 131. However, it is not absolutely necessary for substances derived from a living organism contained in a hybridization solution 9 to be labeled with a radioactive labeling substance, a fluorescent substance and a labeling substance which generates chemiluminescence emission when it contacts a chemiluminescent substrate, and it is sufficient for substances derived from a living organism contained in a hybridization solution 9 to be labeled with at least one kind of labeling substance selected from a group consisting of a radioactive labeling substance, a fluorescent substance and a labeling substance which generates chemiluminescence emission when it contacts a chemiluminescent substrate.

Further, in the above described embodiments, specific binding substances are hybridized with substances derived from a living organism and labeled with a radioactive labeling substance, a fluorescent substance and a labeling substance which generates chemiluminescence emission when it contacts a chemiluminescent substrate. However, it is not absolutely necessary to hybridize substances derived from a living organism with specific binding substances and substances derived from a living organism may be specifically bound with specific binding substances by means of antigen-antibody reaction, receptor-ligand reaction or the like instead of hybridization.

Furthermore, in the above described embodiments, although a number of the substantially circular stimulable phosphor layer regions 12, 17 are regularly formed on one surface of the support 11 of the stimulable phosphor sheet 10, 15 in the same pattern as that of a number of the recesses 3, 123 or the through-holes 133 formed in the substrate 2, 122, 132 of the biochemical analysis unit 1, 121, 131 so that each of them has the same size as that of each of a number of the recesses 3, 123 or the through-holes 133, it is not absolutely necessary to form a number of stimulable phosphor layer regions 12, 17 on one surface of the support 11 of the stimulable phosphor sheet 10, 15 and a stimulable phosphor layer may be continuously formed on one surface of the support 11 of the stimulable phosphor sheet 10, 15.

Moreover, in the above described embodiments, although a number of the substantially circular stimulable phosphor layer regions 12, 17 are regularly formed on one surface of the support 11 of the stimulable phosphor sheet 10, 17 in the same pattern as that of a number of the recesses 3, 123 or the through-holes 133 formed in the substrate 2, 122, 132 of the biochemical analysis unit 1, 121, 131 so that each of them has the same size as that of each of a number of the recesses 3, 123 or the through-holes 133, it is sufficient for a number of the stimulable phosphor layer regions 12, 17 to be formed in the same pattern as that of a number of the recesses 3, 123 or the through-holes 133 formed in the substrate 2, 122, 132 of the biochemical analysis unit 1, 121, 131 and it is not absolutely necessary to regularly form a number of the stimulable phosphor layer regions 12, 17.

Further, in the above described embodiments, although a number of the substantially circular stimulable phosphor layer regions 12, 17 are regularly formed on one surface of the support 11 of the stimulable phosphor sheet 10, 15 in the same pattern as that of a number of the recesses 3, 123 or the through-holes 133 formed in the substrate 2, 122, 132 of the biochemical analysis unit 1, 121, 131 so that each of them has the same size as that of each of a number of the recesses 3, 123 or the through-holes 133, it is not absolutely necessary a number of the stimulable phosphor layer regions 12, 17 to be formed substantially circular and each of the stimulable phosphor layer regions 12, 17 may be formed in an arbitrary shape, for example, a rectangular shape.

Furthermore, in the above described embodiments, although a number of the substantially circular stimulable phosphor layer regions 12, 17 are regularly formed on one surface of the support 11 of the stimulable phosphor sheet 10, 15 in the same pattern as that of a number of the recesses 3, 123 or the through-holes 133 formed in the substrate 2, 122, 132 of the biochemical analysis unit 1, 121, 131 so that each of them has the same size as that of each of a number of the recesses 3, 123 or the through-holes 133, it is not absolutely necessary to form each of the stimulable phosphor layer regions 12, 17 so as to have the same size as that of each of the recesses 3, 123 or the through-holes 133 and the size of each of the stimulable phosphor layer regions 12, 17 may be arbitrarily selected in accordance with the purpose. Preferably, each of the stimulable phosphor layer regions 12, 17 is formed in the stimulable phosphor sheet 10, 15 so as to be equal to or larger than the size of each of the recesses 3, 123 or the through-holes 133.

Moreover, in the above described embodiments, although a number of the stimulable phosphor layer regions 12, 17 of the stimulable phosphor sheet 10, 15 are formed by embedding stimulable phosphor in a number of the recesses 13 formed in the support 11, it is not absolutely necessary to form a number of the stimulable phosphor layer regions 12, 17 by embedding stimulable phosphor in a number of the recesses 13 formed in the support 11. A number of the stimulable phosphor layer regions 12, 17 may be formed by charging or embedding stimulable phosphor in a number of through-holes formed in the support 11 or may be formed on the surface of the support 11.

Further, although the support 11 of the stimulable phosphor sheet 10, 15 is made of stainless steel, it is not absolutely necessary to make the support 11 of stainless steel and the support 11 of the stimulable phosphor sheet 10, 15 may be made of other material. The support 11 of the stimulable phosphor sheet 10, 15 is preferably made of material capable of attenuating radiation energy and light energy but the material for forming the support 11 of the stimulable phosphor sheet 10, 15 is not particularly limited. The support 11 of the stimulable phosphor sheet 10, 15 can be formed of either inorganic compound material or organic compound material and is preferably formed of a metal material, a ceramic material or a plastic material. Illustrative examples of inorganic compound materials usable for forming the support 11 of the stimulable phosphor sheet 10, 15 and capable of attenuating radiation energy and light energy include metals such as gold, silver, copper, zinc, aluminum, titanium, tantalum, chromium, steel, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. High molecular compounds capable of attenuating radiation energy are preferably used as organic compound material for forming the support 11 of the stimulable phosphor sheet 10, 15 and capable of attenuating radiation energy and light energy. Illustrative examples include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4,10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Furthermore, in the embodiment shown in FIGS. 24 and 25, although the absorptive layers 134 are formed of nitrocellulose on the inner surfaces 133a of the through-holes 133 formed in the substrate 132, the surface of each of the absorptive layers 134 may be roughened so as to have a fractal structure similarly to the embodiment shown in FIG. 23.

Moreover, in the embodiment shown in FIG. 23, although the surface of each of the absorptive layers 124 formed on the inner surface 123a of the recesses 133 is roughened so as to have a fractal structure, it is not absolutely necessary to roughen the surface of each of the absorptive layers 124 so as to have a fractal structure and the surface of each of the absorptive layers 124 may be roughened so as to have a multiple projection structure or a micro-pore structure instead of a fractal structure.

Further, although the absorptive layers 4, 134 are formed of nitrocellulose in the embodiment shown in FIGS. 1 and 2 and the embodiment shown in FIGS. 24 and 25 and the absorptive layers 124 are formed of nylon-6, it is not absolutely necessary to form the absorptive layers 4, 124, 134 of nitrocellulose or nylon-6 and the absorptive layers 4, 124, 134 may be formed of other absorptive material. A porous material or a fiber material may be preferably used as the absorptive material for forming the absorptive layers 4, 124, 134 of the biochemical analysis unit 1, 121, 131 and the absorptive regions 4, 124, 134 of the biochemical analysis unit 1, 121, 131 may be formed by combining a porous material and a fiber material. A porous material for forming the absorptive layers 4, 124, 134 of the biochemical analysis unit 1, 121, 131 may be any type of an organic material or an inorganic material and may be an organic/inorganic composite material. An organic porous material used for forming the absorptive layers 4, 124, 134 of the biochemical analysis unit 1, 121, 131 is not particularly limited but a carbon porous material such as an activated carbon or a porous material capable of forming a membrane filter can be preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluoride such as polyvinylidene fluoride, polytetrafluoride; and copolymers or composite materials thereof An inorganic porous material used for forming the absorptive layers 4, 124, 134 of the biochemical analysis unit 1, 121, 131 is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof. A fiber material used for forming the absorptive regions 4, 124, 134 of the biochemical analysis unit 1, 121, 131 is not particularly limited. Illustrative examples of fiber materials preferably usable in the present invention include nylons such as nylon-6, nylon-6,6, nylon-4,10; and cellulose derivatives such as nitrocellulose, acetyl cellulose, butyric-acetyl cellulose.

Furthermore, in the above described embodiments, biochemical analysis data are produced by reading radiation data of a radioactive labeling substance recorded in a number of the stimulable phosphor layer regions 12 formed in the stimulable phosphor sheet 10 and fluorescence data of a fluorescent substance such as a fluorescent dye recorded in a number of the absorptive layers 4, 124, 134 formed in the biochemical analysis unit 1 using the scanner shown in FIGS. 7 to 14. However, it is not absolutely necessary to produce biochemical analysis data by reading radiation data of a radioactive labeling substance and fluorescence data of a fluorescent substance using a single scanner and biochemical analysis data may be produced by reading radiation data of a radioactive labeling substance and fluorescence data of a fluorescent substance using separate scanners.

Moreover, in the above described embodiments, although biochemical analysis data are produced by reading radiation data of a radioactive labeling substance recorded in a number of the stimulable phosphor layer regions 12 formed in the stimulable phosphor sheet 10 and fluorescence data of a fluorescent substance such as a fluorescent dye recorded in a number of the absorptive layers 4, 124, 134 formed in the biochemical analysis unit 1, 121, 131 using the scanner shown in FIGS. 7 to 14 and biochemical analysis data are produced by reading chemiluminescence data recorded in a number of the stimulable phosphor layer regions 17 formed in the stimulable phosphor sheet 15 using the scanner shown in FIGS. 20 to 22. it is not absolutely necessary to read radiation data recorded in a number of the stimulable phosphor layer regions 12 formed in the stimulable phosphor sheet 10 and fluorescence data recorded in a number of the absorptive layers 4, 124, 134 formed in the biochemical analysis unit 1, 121, 131 using the scanner shown in FIGS. 7 to 14 and to read chemiluminescence data recorded in a number of the stimulable phosphor layer regions 17 formed in the stimulable phosphor sheet 15 using the scanner shown in FIGS. 20 to 22 and any scanner constituted so as to scan a number of the stimulable phosphor layer regions 12, 15 of the stimulable phosphor layer sheet 10, 17 and a number of the absorptive layers 4, 124, 134 of the biochemical analysis unit 1, 121, 131, thereby exciting stimulable phosphor contained in a number of the stimulable phosphor layer regions 12, 17 and a fluorescent substance contained in a number of the absorptive layers 4, 124, 134 with a laser beam 24 or other stimulating ray may be used for reading radiation data of a radioactive labeling substance and fluorescence data of a fluorescent substance.

Further, in the above described embodiments, although the scanner shown in FIGS. 7 to 14 includes the first laser stimulating ray source 21, the second laser stimulating ray source 22 and the third laser stimulating ray source 23 and the scanner shown in FIGS. 20 to 22 includes the first laser stimulating ray source 21, the second laser stimulating ray source 22 and the fourth laser stimulating ray source 55, it is not absolutely necessary for the scanner to include three laser stimulating ray sources.

Furthermore, in the above described embodiments, chemiluminescence data of a labeling substance which generates chemiluminescence emission when it contacts a chemiluminescent substrate recorded in the absorptive layers 4, 124, 134 formed on the inner surfaces 3a, 123a, 133a of a number of the recesses 3, 123 or the through-holes 133 of the biochemical analysis unit 1, 121, 131 are read by the data producing system shown in FIGS. 15 to 18 which can read fluorescence data. However, it is not absolutely necessary to produce biochemical analysis data by reading chemiluminescence data using a data producing system which can also read fluorescence data, and in the case where the data producing system is used for reading only chemiluminescence data of a labeling substance which generates chemiluminescence emission when it contacts a chemiluminescent substrate recorded in a number of the absorptive layers 4, 124, 134 formed on the inner surfaces 3a, 123a, 133a of a number of the recesses 3, 123 or the through-holes 133 of the biochemical analysis unit 1, 121, the light emitting diode stimulating ray source 100, the filter 101, the filter 102 and the diffusion plate 102 can be omitted from the data producing system.

Moreover, in the above described embodiments, each of the scanner shown in FIGS. 7 to 14 and the scanner shown in FIGS. 20 to 22 is constituted so that all of the stimulable phosphor layer regions 12, 17 of the stimulable phosphor sheet 10, 15 or all of the absorptive layers 4, 124, 134 of the biochemical analysis unit 1, 121, 131 are scanned with a laser beam 24 to excite stimulable phosphor or a fluorescent substance such as a fluorescent dye by moving the optical head 35 using a scanning mechanism in the main scanning direction indicated by the arrow X direction and the sub-scanning direction indicated by the arrow Y in FIG. 13. However, all of the stimulable phosphor layer regions 12, 17 of the stimulable phosphor sheet 10, 15 or all of the absorptive layers 4, 124, 134 of the biochemical analysis unit 1, 121, 131 may be scanned with a laser beam 24 to excite stimulable phosphor or a fluorescent substance such as a fluorescent dye by moving the stage 40 in the main scanning direction indicated by the arrow X direction and the sub-scanning direction indicated by the arrow Y in FIG. 13, while holding the optical head 35 stationary, or moving the optical head 35 in the main scanning direction indicated by the arrow X direction or the sub-scanning direction indicated by the arrow Y in FIG. 13 and moving the stage 40 in the sub-scanning direction indicated by the arrow Y or the main scanning direction indicated by the arrow X in FIG. 13.

Further, each of the scanner shown in FIGS. 7 to 14 and the scanner shown in FIGS. 20 to 22 employs the photomultiplier 50 as a light detector to photoelectrically detect fluorescence emission or stimulated. However, it is sufficient for the light detector used in the present invention to be able to photoelectrically detect fluorescence emission or stimulated emission and it is possible to employ a light detector such as a line CCD or a two-dimensional CCD instead of the photomultiplier 50.

Furthermore, a solution containing specific binding substances such as cDNAs are spotted using the spotting device 5 including an injector 6 and a CCD camera 7 so that when the tip end portion of the injector 6 and the center of the recess 3, 123 or the through-hole 133 into which a solution containing specific binding substances is to be spotted are determined to coincide with each other as a result of viewing them using the CCD camera 7, the solution containing the specific binding substances such as cDNA is spotted from the injector 6. However, the solution containing specific binding substances such as cDNAs can be spotted by detecting the positional relationship between a number of the absorptive recesses 3, 123 or the through-holes 133 formed in the biochemical analysis unit 1, 121, 131 and the tip end portion of the injector 6 in advance and two-dimensionally moving the biochemical analysis unit 1, 121, 131 or the tip end portion of the injector 6 so that the tip end portion of the injector 6 coincides with each of the recesses 3, 123 or the through-holes 133

According to the present invention, it is possible to provide a biochemical analysis unit which can prevent noise caused by the scattering of electron beams (β rays) released from a radioactive labeling substance from being generated in biochemical analysis data even in the case of forming spot-like regions of specific binding substances on a substrate at high density, which can specifically bind with a substance derived from a living organism and whose sequence, base length, composition and the like are known, specifically binding the specific binding substances contained in the spot-like regions with a substance derived from a living organism and labeled with a radioactive substance to selectively label the spot-like regions with a radioactive substance, thereby obtaining a biochemical analysis unit, superposing the thus obtained biochemical analysis unit and a stimulable phosphor layer, exposing the stimulable phosphor layer to the radioactive labeling substance, irradiating the stimulable phosphor layer with a stimulating ray to excite the stimulable phosphor, photoelectrically detecting the stimulated emission released from the stimulable phosphor layer to produce biochemical analysis data, and analyzing the substance derived from a living organism.

Furthermore, according to the present invention, it is possible to provide a biochemical analysis unit which can prevent noise caused by the scattering of chemiluminescence emission from being generated in biochemical analysis data even in the case of forming spot-like regions of specific binding substances on a substrate at high density, which can specifically bind with a substance derived from a living organism and whose sequence, base length, composition and the like are known, specifically binding the specific binding substance contained in the spot-like regions with a substance derived from a living organism and labeled with a labeling substance which generates chemiluminescence emission when it contacts a chemiluminescent substrate to selectively label the spot-like regions with a labeling substance which generates chemiluminescence emission when it contacts a chemiluminescent substrate, thereby obtaining a biochemical analysis unit, bringing the thus obtained biochemical analysis unit into contact with a chemiluminescent substrate, thereby causing it to release chemiluminescence emission, superposing the biochemical analysis unit releasing chemiluminescence emission and a stimulable phosphor layer, exposing the stimulable phosphor layer to the chemiluminescence emission, irradiating the thus exposed stimulable phosphor layer with a stimulating ray to excite the stimulable phosphor, photoelectrically detecting the stimulated emission released from the stimulable phosphor layer to produce biochemical analysis data, and analyzing the substance derived from a living organism.

Further, according to the present invention, it is possible to provide a biochemical analysis unit which can prevent noise caused by the scattering of chemiluminescence emission released from a labeling substance which generates chemiluminescence emission when it contacts a chemiluminescent substrate or fluorescence emission released from a fluorescent substance from being generated in biochemical analysis data even in the case of forming spot-like regions of specific binding substances on a substrate at high density, which can specifically bind with a substance derived from a living organism and whose sequence, base length, composition and the like are known, specifically binding the specific binding substance contained in the spot-like regions with a substance derived from a living organism and labeled with, in addition to a radioactive labeling substance or instead of a radioactive labeling substance, a labeling substance which generates chemiluminescence emission when it contacts a chemiluminescent substrate and/or a fluorescent substance to selectively label the spot-like specific binding substances therewith, thereby obtaining a biochemical analysis unit, photoelectrically detecting chemiluminescence emission or fluorescence emission released from the biochemical analysis unit to produce biochemical analysis data, and analyzing the substance derived from a living organism.

1. A biochemical analysis unit including a substrate made of a material capable of attenuating radiation energy and/or light energy and formed with a plurality of holes spaced apart from each other, a plurality of absorptive layers being formed on inner surfaces of the holes.

2. A biochemical analysis unit including a substrate made of a material capable of attenuating radiation energy and/or light energy and formed with a plurality of holes spaced apart from each other, a plurality of absorptive layers being formed on inner surfaces of the plurality of holes formed in the substrate and being selectively labeled with at least one kind of a labeling substance selected from a group consisting a radioactive labeling substance, a fluorescent substance and a labeling substance which generates chemiluminescence emission when it contacts a chemiluminescent substrate.

3. A biochemical analysis unit in accordance with claim 2 wherein the substance derived from a living organism is specifically bound with specific binding substances by a reaction selected from a group consisting of hybridization, antigen-antibody reaction and receptor-ligand reaction.

4. A biochemical analysis unit in accordance with claim 1 wherein the plurality of holes are formed by recesses.

5. A biochemical analysis unit in accordance with claim 2 wherein the plurality of holes are formed by recesses.

6. A biochemical analysis unit in accordance with claim 1 wherein the plurality of holes are formed by through-holes.

7. A biochemical analysis unit in accordance with claim 2 wherein the plurality of holes are formed by through-holes.

8. A biochemical analysis unit in accordance with claim 1 wherein each of the absorptive layers is formed of a porous material.

9. A biochemical analysis unit in accordance with claim 2 wherein each of the absorptive layers is formed of a porous material.

10. A biochemical analysis unit in accordance with claim 8 wherein each of the absorptive layers is formed of a carbon porous material or a porous material capable of forming a membrane filter.

11. A biochemical analysis unit in accordance with claim 9 wherein each of the absorptive layers is formed of a carbon porous material or a porous material capable of forming a membrane filter.

12. A biochemical analysis unit in accordance with claim 1 wherein each of the absorptive layers is formed of a fiber material.

13. A biochemical analysis unit in accordance with claim 2 wherein each of the absorptive layers is formed of a fiber material.

14. A biochemical analysis unit in accordance with claim 1 wherein the plurality of absorptive layers are formed by surface-processing the substrate with a surface modifying reforming agent.

15. A biochemical analysis unit in accordance with claim 2 wherein the plurality of absorptive layers are formed by surface-processing the substrate with a surface modifying reforming agent.

16. A biochemical analysis unit in accordance with claim 14 wherein the plurality of absorptive layers are formed by surface-processing the substrate with a silane coupling agent.

17. A biochemical analysis unit in accordance with claim 15 wherein the plurality of absorptive layers are formed by surface-processing the substrate with a silane coupling agent.

18. A biochemical analysis unit in accordance with claim 1 wherein the surfaces of the plurality of absorptive layers are roughened.

19. A biochemical analysis unit in accordance with claim 2 wherein the surfaces of the plurality of absorptive layers are roughened.

20. A biochemical analysis unit in accordance with claim 18 wherein the surface of each of the absorptive layers is roughened so as to have a fractal structure.

21. A biochemical analysis unit in accordance with claim 19 wherein the surface of each of the absorptive layers is roughened so as to have a fractal structure.

22. A biochemical analysis unit in accordance with claim 1 wherein the substrate is formed with 10 or more holes.

23. A biochemical analysis unit in accordance with claim 2 wherein the substrate is formed with 10 or more holes.

24. A biochemical analysis unit in accordance with claim 22 wherein the substrate is formed with 1,000 or more holes.

25. A biochemical analysis unit in accordance with claim 23 wherein the substrate is formed with 1,000 or more holes.

26. A biochemical analysis unit in accordance with claim 24 wherein the substrate is formed with 10,000 or more holes.

27. A biochemical analysis unit in accordance with claim 25 wherein the substrate is formed with 10,000 or more holes.

28. A biochemical analysis unit in accordance with claim 1 wherein each of the plurality of holes formed in the substrate has a size of less than 5 mm².

29. A biochemical analysis unit in accordance with claim 2 wherein each of the plurality of holes formed in the substrate has a size of less than 5 mm².

30. A biochemical analysis unit in accordance with claim 28 wherein each of the plurality of holes formed in the substrate has a size of less than 1 mm².

31. A biochemical analysis unit in accordance with claim 29 wherein each of the plurality of holes formed in the substrate has a size of less than 1 mm².

32. A biochemical analysis unit in accordance with claim 30 wherein each of the plurality of holes formed in the substrate has a size of less than 0.1 mm².

33. A biochemical analysis unit in accordance with claim 31 wherein each of the plurality of holes formed in the substrate has a size of less than 0.1 mm².

34. A biochemical analysis unit in accordance with claim 1 wherein the plurality of holes are formed in the substrate at a density of 10 or more per cm².

35. A biochemical analysis unit in accordance with claim 2 wherein the plurality of holes are formed in the substrate at a density of 10 or more per cm².

36. A biochemical analysis unit in accordance with claim 34 wherein the plurality of holes are formed in the substrate at a density of 1,000 or more per cm².

37. A biochemical analysis unit in accordance with claim 35 wherein the plurality of holes are formed in the substrate at a density of 1,000 or more per cm².

38. A biochemical analysis unit in accordance with claim 36 wherein the plurality of holes are formed in the substrate at a density of 10,000 or more per cm².

39. A biochemical analysis unit in accordance with claim 37 wherein the plurality of holes are formed in the substrate at a density of 10,000 or more per cm².

40. A biochemical analysis unit in accordance with claim 1 wherein the material capable of attenuating radiation energy and/or light energy has a property of reducing the energy of radiation and/or the energy of light to ⅕ or less when the radiation and/or light travels in the material by a distance equal to that between neighboring absorptive layers.

41. A biochemical analysis unit in accordance with claim 2 wherein the material capable of attenuating radiation energy and/or light energy has a property of reducing the energy of radiation and/or the energy of light to ⅕ or less when the radiation and/or light travels in the material by a distance equal to that between neighboring absorptive layers.

42. A biochemical analysis unit in accordance with claim 40 wherein the material capable of attenuating radiation energy and/or light energy has a property of reducing the energy of radiation and/or the energy of light to 1/10 or less when the radiation and/or light travels in the material by a distance equal to that between neighboring absorptive layers.

43. A biochemical analysis unit in accordance with claim 41 wherein the material capable of attenuating radiation energy and/or light energy has a property of reducing the energy of radiation and/or the energy of light to 1/10 or less when the radiation and/or light travels in the material by a distance equal to that between neighboring absorptive layers.

44. A biochemical analysis unit in accordance with claim 42 wherein the material capable of attenuating radiation energy and/or light energy has a property of reducing the energy of radiation and/or the energy of light to 1/100 or less when the radiation and/or light travels in the material by a distance equal to that between neighboring absorptive layers.

45. A biochemical analysis unit in accordance with claim 43 wherein the material capable of attenuating radiation energy and/or light energy has a property of reducing the energy of radiation and/or the energy of light to 1/100 or less when the radiation and/or light travels in the material by a distance equal to that between neighboring absorptive layers.

46. A biochemical analysis unit in accordance with claim 1 wherein the substrate is formed of a material selected from a group consisting of a metal material, a ceramic material and a plastic material.

47. A biochemical analysis unit in accordance with claim 2 wherein the substrate is formed of a material selected from a group consisting of a metal material, a ceramic material and a plastic material.
